(19) United States
(12) Patent Application Publication
(10) Pub. No.: US 2024/0257922 A1
(43) Pub. Date: Aug. 1, 2024

(54) MODEL GENERATION METHOD, DATA PRESENTATION METHOD, DATA GENERATION METHOD, INFERENCE METHOD, MODEL GENERATION DEVICE, DATA PRESENTATION DEVICE, DATA GENERATION DEVICE, AND INFERENCE DEVICE

(71) Applicants: OMRON Corporation, Kyoto-shi, KYOTO (JP); Inter-University Research Institute Corporation High Energy Accelerator Research Organization, Tsukuba-shi, Ibaraki (JP)

(72) Inventors: Tatsunori TANIAI, Bunkyo-ku, TOKYO (JP); Yoshitaka USHIKU, Bunkyo-ku, TOKYO (JP); Naoya CHIBA, Bunkyo-ku, TOKYO (JP); Yuta SUZUKI, Tsukuba-shi, IBARAKI (JP); Kanta ONO, Tsukuba-shi, IBARAKI (JP)

(21) Appl. No.: 18/686,602

(22) PCT Filed: Aug. 17, 2022
(86) PCT No.: PCT/JP2022/031003

§ 371 Date: Feb. 26, 2024

(30) Foreign Application Priority Data
Sep. 27, 2021 (JP) 2021-157205

(51) Int. Cl. G16C 20/70; G16C 20/50

(52) U.S. Cl. CPC G16C 20/70; G16C 20/50

(57) ABSTRACT

A model generation method according to one aspect of the present invention acquires first data and second data regarding a crystal structure of a material, and performs machine learning for a first encoder and a second encoder by using the first data and the second data. The second data indicates a property of the material with an index different from that of the first data. The first encoder is configured to convert the first data into a first feature vector, and the second encoder is configured to convert the second data into a second feature vector. The dimension of the first feature vector is the same as the dimension of the second feature vector. In machine learning, the first encoder and the second encoder are trained so that the values of the feature vectors of the positive samples are positioned close to each other, and the feature vector of the negative sample is positioned far from the feature vector of the positive sample.

### TECHNICAL FIELD

The present invention relates to a model generation method, a data presentation method, a data generation method, an estimation method, a model generation device, a data presentation device, a data generation device, and an estimation device.

### BACKGROUND ART

In recent years, information processing technology including machine learning has been utilized for material development. This field is called Materials Informatics (MI), and contributes greatly to the efficiency of new material development. As a typical method of estimating a characteristic of a material by information processing, a method using first-principles calculations disclosed in Non-Patent Document 1 and the like is known. The first-principles calculations are methods of calculating the state of electrons in a substance based on the Schrodinger equation of quantum mechanics. According to the first-principles calculations, a characteristic of a substance can be estimated based on a state of an electron calculated under various conditions.

### PRIOR ART DOCUMENTS

### Non-Patent Document

Non-Patent Document 1: Masanori Kohyama, “Frontier and Future of Computational Materials Science: Applications to Materials Interfaces”, Journal of the Surface Finishing Society of Japan, Vol. 2013, 64, No. 10, p. 524-530.

### SUMMARY OF THE INVENTION

### Problems to be Solved by the Invention

The present inventors have found that the conventional method for MI has the following problems. That is, since calculating the Schrodinger equation in the actual material (multibody electron system) is extremely complicated, approximate calculations using density functional methods or the like is used. The accuracy depends on the approximate calculations employed. With the capability of a general computer at present, it is difficult to execute highly accurate first-principles calculations in a realistic time, and hence it is difficult to estimate the characteristic as the target material becomes more complicated. Therefore, the development is in progress for a method in which machine learning is performed by providing knowledge, such as a characteristic related to a known material and a feature portion of its crystal structure, as correct information to generate a trained inference model, and new knowledge, such as a composition and a characteristic of a new material, is obtained using the generated trained inference model. However, with such a method, it is difficult to accurately obtain new knowledge within a range in which correct information is not provided. In addition, it is extremely costly to provide correct information for all known materials. It is thus difficult to obtain new knowledge with high accuracy at low cost by the machine learning method that provides correct information on a known material.

The present invention has been made in view of such circumstances in one aspect, and an object of the present invention is to provide a technique for obtaining new knowledge related to a material at low cost and a method for utilizing the technique.

### Means for Solving the Problems

To solve the problems described above, the present invention adopts the following configuration.

That is, a model generation method according to one aspect of the present invention is an information processing method including the steps of: a step of acquiring, by a computer, first data and second data regarding a crystal structure of a material; and a step of performing, by the computer, machine learning for a first encoder and a second encoder by using the acquired first data and second data. The second data is configured to indicate a property of the material with an index different from that of the first data. The acquired first data and second data include a positive sample and a negative sample. The positive sample includes a combination of the first data and the second data for the same material. The negative sample includes at least one of first data and second data for a material different from the material of the positive sample. The first encoder is configured to convert first data into a first feature vector, and the second encoder is configured to convert the second data into a second feature vector. The dimension of the first feature vector is the same as the dimension of the second feature vector. The machine learning for the first encoder and the second encoder is configured by training the first encoder and the second encoder so that values of the first feature vector and the second feature vector calculated from the first data and the second data of the positive sample are positioned close to each other, and at least one value of a first feature vector and a second feature vector calculated from at least one of the first data and the second data of the negative sample is positioned far from at least one value of the first feature vector and the second feature vector calculated from the positive sample.

In an experimental example to be described later, trained encoders that map a plurality of different types of data regarding the crystal structure onto the same dimensional feature space were generated by machine learning, respectively. In this machine learning, each encoder was trained so that feature vectors of various types of data (positive sample) for the same material were positioned close to each other on the feature space, and a feature vector of data (negative sample) for a different material was positioned far from the feature vectors of the positive sample. Then, when various types of data were mapped onto the feature space by using each generated trained encoder, various types of data for each material with similar features were mapped to the proximity range on the feature space. The result of this experimental example showed the following: according to each trained encoder generated by such machine learning, it is possible to evaluate the similarity of materials, based on the positional relationship on the feature space and to accurately acquire new knowledge of the material from the evaluation result without providing knowledge of the composition, characteristic, and the like of the known material.

As described above, in the case of generating a highly accurate trained model that directly derives the property of the material from the data regarding the crystal structure, it takes a lot of time and effort to provide correct information for all known materials. In contrast, in the model generation method according to this configuration, it is possible to prepare a positive sample and a negative sample to be used for machine learning depending on whether or not the materials are the same, eliminating the time and effort for providing correct information for all known materials. Thus, in the model generation method according to this configuration, the trained encoders (the first encoder and the second encoder) that map the first data and the second data onto the feature space as described above can be generated at low cost. As a result, with each trained encoder generated, new knowledge related to the material can be obtained at low cost. In addition, since it is not necessary to provide correct information, it is possible to prepare a large number of positive samples and negative samples used for machine learning at low cost. Therefore, a trained encoder for accurately obtaining new knowledge related to a material can be generated at low cost.

The model generation method according to one aspect may further include a step of performing, by the computer, machine learning for a first decoder. The machine learning for the first decoder may be configured by training the first decoder so that a result of restoring the first data by the first decoder from a first feature vector, calculated from the first data by using the first encoder, matches the first data. With this configuration, it is possible to generate the trained first decoder that has acquired the ability to restore the first data. By using the generated trained first decoder and trained second encoder, first data can be generated from the second data for a material known in the second data but unknown in the first data.

The model generation method according to one aspect may further include a step of performing, by the computer, machine learning for a second decoder. The machine learning for the second decoder may be configured by training the second decoder so that a result of restoring the second data by the second decoder from a second feature vector, calculated from the second data by using the second encoder, matches the second data. With this configuration, it is possible to generate the trained second decoder that has acquired the ability to restore the second data. By using the generated trained second decoder and trained first encoder, second data can be generated from the first data for a material known in the first data but unknown in the second data.

The model generation method according to one aspect may further include a step of performing, by the computer, machine learning for an estimator. In the step of acquiring the first data and the second data, the computer may further acquire correct information indicating the characteristic of the material. The machine learning for the estimator may be configured by training the estimator using the first encoder and the second encoder so that a result of estimating the characteristic of the material from at least one of the first feature vector and the second feature vector, calculated from the first data and the second data acquired, matches the correct information.

With this configuration, a trained estimator for estimating a characteristic of a material can be generated. In this configuration, correct information may be provided for all materials for learning, but information regarding the similarity of materials is included in a feature space where mapping is performed by each trained encoder. Since the estimator is configured to estimate the characteristic of the material from the feature vector on the feature space, the information can be used at the time of estimating the characteristic of the material. It is thus possible to generate a trained estimator capable of accurately estimating the characteristic of the material without preparing correct information for all materials. Therefore, with this configuration, a trained estimator capable of accurately estimating a characteristic of a material can be generated at low cost.

In the model generation method according to one aspect, the first data indicates information regarding a local structure of the crystal of the material, and the second data indicates information regarding the periodicity of the crystal structure of the material. In this configuration, as the first data, data indicating the property of the material based on a local perspective of the crystal structure is adopted. As the second data, data indicating the property of the material based on an overall overhead perspective is adopted. As a result, in the feature space where mapping is performed by the generated trained encoder, the similarity of materials can be evaluated from both the local and overhead perspectives, and new knowledge of the material can be accurately acquired from the evaluation result.

In the model generation method according to one aspect, the first data may include at least one of three-dimensional atomic position data, Raman spectroscopy data, nuclear magnetic resonance spectroscopy data, infrared spectroscopy data, mass spectrometry data, and X-ray absorption spectroscopy data as the data indicating the property of the material based on a local perspective of the crystal structure. Alternatively, the first data may include three-dimensional atomic position data, and the three-dimensional atomic position data may be configured to express a state of an atom in the material by at least one of a probability density function, a probability distribution function, and a probability mass function. With these configurations, it is possible to appropriately prepare the first data indicating the property of the material based on a local perspective of the crystal structure.

In the model generation method according to one aspect, the second data may include at least one of X-ray diffraction data, neutron diffraction data, electron beam diffraction data, and total scattering data as data indicating a property of a material based on an overall overhead perspective. With this configuration, it is possible to appropriately prepare the second data indicating the property of the material based on the overall overhead perspective.

The mode of the present invention is not limited to the model generation method configured to execute the series of information processing by a computer. One aspect of the present invention may be a data processing method using a trained machine learning model generated by the model generation method according to any of the above modes.

For example, a data presentation method according to one aspect of the present invention is an information processing method including the steps of: acquiring, by a computer, at least one of first data and second data regarding a crystal structure of each of a plurality of target materials; converting, by the computer, at least one of the first data and second data acquired of each of the target materials into at least one of a first feature vector and a second feature vector by using at least one of a trained first encoder and a trained second encoder; mapping, by the computer, each value of at least one of the first feature vector and the second feature vector obtained of each of the target materials onto a space; and outputting, by the computer, each of the values of at least one of the first feature vector and the second feature vector of each of the target materials mapped onto the space. The trained first encoder and the trained second encoder may be generated by machine learning using the first data and the second data for learning in any of the above model generation methods.

In the data presentation method according to one aspect, in the step of mapping, the computer converts each of the values of at least one of the first feature vector and the second feature vector obtained of each of the target materials into a low dimension to maintain a positional relationship of each of the values, and then maps each of the values converted onto a space. In the step of outputting each of the values, the computer outputs each of the values converted of at least one of the first feature vector and the second feature vector of each of the target materials. With this configuration, when each value of the feature vector is output to obtain new knowledge of the material, the value is converted into a low dimension to maintain the positional relationship of each of the values, whereby it is possible to improve the efficiency of the output resources (e.g., saving space in an information output range, improving visibility, etc.) while reducing the impact on the information regarding the similarity of the material.

Further, for example, a data generation method according to one aspect of the present invention is an information processing method to generate second data from first data. The first data and the second data relate to the crystal structure of the target material. The second data is configured to indicate a property of the material with an index different from that of the first data. The data generation method includes the steps of: acquiring, by a computer, first data for the target material; converting, by the computer, the first data acquired of the target material into a first feature vector by using a trained first encoder; and restoring, by the computer, the second data from at least one of a value of the first feature vector obtained by the conversion and a value in a proximity of the value by using a trained decoder. The trained first encoder may be generated by machine learning using the first data and the second data for learning together with the second encoder in any of the above model generation methods. The trained decoder (second decoder) may be generated by machine learning using the second data for learning in any of the above model generation methods. The first data may indicate information regarding the local structure of the crystal of the target material, and the second data may indicate information regarding the periodicity of the crystal structure of the target material.

Further, for example, a data generation method according to one aspect of the present invention is an information processing method to generate first data from second data. The first data and the second data relate to the crystal structure of the target material. The second data is configured to indicate a property of the material with an index different from that of the first data. The data generation method includes the steps of: *acquiring, by a computer, second data for the target material; converting, by the computer, the second data acquired of the target material into a second feature vector by using a trained second encoder; and restoring first data from at least one of a value of the second feature vector obtained by the conversion and a value in a proximity of the value by using a trained decoder to generate the first data. The trained second encoder may be generated by machine learning using the first data and the second data for learning together with the first encoder in any of the above model generation methods. The trained decoder (first decoder) may be generated by machine learning using the first data for learning in any of the above model generation methods. The first data may indicate information regarding the local structure of the crystal of the target material, and the second data may indicate information regarding the periodicity of the crystal structure of the target material.

Further, for example, an estimation method according to one aspect of the present invention is an information processing method including the steps of: acquiring, by a computer, at least one of first data and second data regarding a crystal structure of a target material; converting, by the computer, at least one of the first data and second data acquired into at least one of a first feature vector and a second feature vector by using at least one of a trained first encoder and a trained second encoder; and estimating, by the computer, a characteristic of the target material from a value of at least one of the obtained first feature vector and second feature vector by using a trained estimator. The trained first encoder and the trained second encoder may be generated by machine learning using the first data and the second data for learning in any of the above model generation methods. The trained estimator may be generated by machine learning further using correct information indicating the characteristic of the material for learning in any of the above model generation methods.

Further, as another mode of the information processing method according to each of the above modes, one aspect of the present invention may be an information processing device that achieves all or part of the above configurations, may be an information processing system, may be a program, or may be a storage medium in which such a program is stored and which can be read by a computer, another device, a machine, or the like. Here, the computer-readable storage medium is a medium that accumulates information such as a program by electrical, magnetic, optical, mechanical, or chemical action.

For example, a model generation device according to one aspect of the present invention is an information processing device including: a learning data acquisition unit configured to acquire first data and second data regarding a crystal structure of a material; and a machine learning unit configured to perform machine learning for a first encoder and a second encoder using the acquired first data and second data.

Further, for example, a data presentation device according to one aspect of the present invention is information processing device including: a target data acquisition unit configured to acquire at least one of first data and second data regarding a crystal structure of each of a plurality of target materials; a conversion unit configured to acquire at least one of a first feature vector and a second feature vector by performing at least one of a process of converting the first data into a first feature vector, using a trained first encoder, and a process of converting the second data into a second feature vector, using a trained second encoder; and an output processing unit configured to map each of values of at least one of the first feature vector and the second feature vector of each of the target materials obtained onto a space and output each of the value of at least one of the first feature vector and the second feature vector of each of the target materials mapped onto the space.

Further, for example, a data generation device according to one aspect of the present invention is an information processing device configured to generate second data from first data. The data generation device includes: a target data acquisition unit configured to acquire, by a computer, first data for the target material; a conversion unit configured to convert the first data acquired of the target material into a first feature vector by using a trained first encoder; and a restoration unit configured to restore second data from at least one of a value of the first feature vector obtained by the conversion and a value in a proximity of the value by using a trained decoder to generate the second data.

Further, for example, a data generation device according to one aspect of the present invention is an information processing device configured to generate first data from second data. The data generation device includes: a target data acquisition unit configured to acquire, by a computer, second data for the target material; a conversion unit configured to convert the second data acquired of the target material into a second feature vector by using a trained second encoder; and a restoration unit configured to restore first data from at least one of a value of the second feature vector obtained by the conversion and a value in a proximity of the value by using a trained decoder to generate the first data.

Further, for example, an estimation device according to one aspect of the present invention is an information processing device including: a target data acquisition unit configured to acquire at least one of first data and second data regarding a crystal structure of a target material; a conversion unit configured to convert at least one of the first data and second data acquired into at least one of a first feature vector and a second feature vector by using at least one of a trained first encoder and a trained second encoder; and an estimation unit configured to estimate a characteristic of the target material from a value of at least one of the obtained first feature vector and second feature vector by using a trained estimator.

### Effect of the Invention

According to the present invention, it is possible to provide a technique for obtaining new knowledge related to a material at low cost and a method for utilizing the technique.

### BRIEF DESCRIPTION OF THE DRAWINGS

FIG. 1 schematically illustrates an example of a scene to which the present invention is applied.

FIG. 2 schematically illustrates an example of a hardware configuration of a model generation device according to an embodiment.

FIG. 3 schematically illustrates an example of a hardware configuration of a data processing device according to the embodiment.

FIG. 4 schematically illustrates an example of the software configuration of the model generation device according to the embodiment.

FIG. 5A schematically illustrates an example of the course of machine learning for a first decoder by the model generation device according to the embodiment.

FIG. 5B schematically illustrates an example of the course of machine learning for a second decoder by the model generation device according to the embodiment.

FIG. 5C schematically illustrates an example of the course of machine learning for an estimator by the model generation device according to the embodiment.

FIG. 6 schematically illustrates an example of the software configuration of the data processing device according to the embodiment.

FIG. 7A schematically illustrates an example of the course of a data presentation process by the data processing device according to the embodiment.

FIG. 7B schematically illustrates an example of the course of a data generation process by the data processing device according to the embodiment.

FIG. 7C schematically illustrates an example of the course of the data generation process by the data processing device according to the embodiment.

FIG. 7D schematically illustrates an example of the course of an estimation process by the data processing device according to the embodiment.

FIG. 8 is a flowchart illustrating an example of a processing procedure for the model generation device according to the embodiment.

FIG. 9 is a flowchart illustrating an example of a process procedure related to a data presentation method of the data processing device according to the embodiment.

FIG. 10A is a flowchart illustrating an example of a process procedure related to a data generation method of the data processing device according to the embodiment.

FIG. 10B is a flowchart illustrating an example of the process procedure related to the data generation method of the data processing device according to the embodiment.

FIG. 11 is a flowchart illustrating an example of a process procedure related to an estimation method of the data processing device according to the embodiment.

FIG. 12 schematically illustrates an example of a configuration of an encoder according to another mode.

FIG. 13 illustrates a result of checking a range in which elements corresponding to materials containing the respective elements of the periodic table are present in a data distribution on a feature space created by an experimental example.

FIG. 14A illustrates a result of color-coding each element according to a value (eV) of a physical characteristic (energy above the hull) in the data distribution on the feature space created by the experimental example.

FIG. 14B illustrates a result of color-coding each element according to a value (eV) of a physical characteristic (band gap) in the data distribution on the feature space created by the experimental example.

FIG. 14C illustrates a result of color-coding each element according to a value (T) of a physical characteristic (magnetization) in the data distribution on the feature space created by the experimental example.

FIG. 15A illustrates a composition of a material used for a query in the experimental example.

FIG. 15B illustrates a composition of a material extracted in the closest proximity on the feature space to the query illustrated in FIG. 15A.

FIG. 15C illustrates a composition of a material extracted in the second proximity on the feature space to the query illustrated in FIG. 15A.

FIG. 16A illustrates a composition of a material used for a query in the experimental example.

FIG. 16B illustrates a composition of a material extracted in the closest proximity on the feature space to the query illustrated in FIG. 16A.

FIG. 16C illustrates a composition of a material extracted in the second proximity on the feature space to the query illustrated in FIG. 16A.

### MODE FOR CARRYING OUT THE INVENTION

An embodiment according to one aspect of the present invention (hereinafter also referred to as “the present embodiment”) will be described below with reference to the drawings. However, the present embodiment described below is merely an example of the present invention in all respects. It goes without saying that various improvements and modifications can be made without departing from the scope of the present invention. That is, in practicing the present invention, a specific configuration according to the embodiment may be adopted as appropriate. Although data appearing in the present embodiment is described in a natural language, the data is designated, more specifically, in a pseudo-language, a command, a parameter, a machine language, or the like that can be recognized by a computer.

### § 1 Application Example

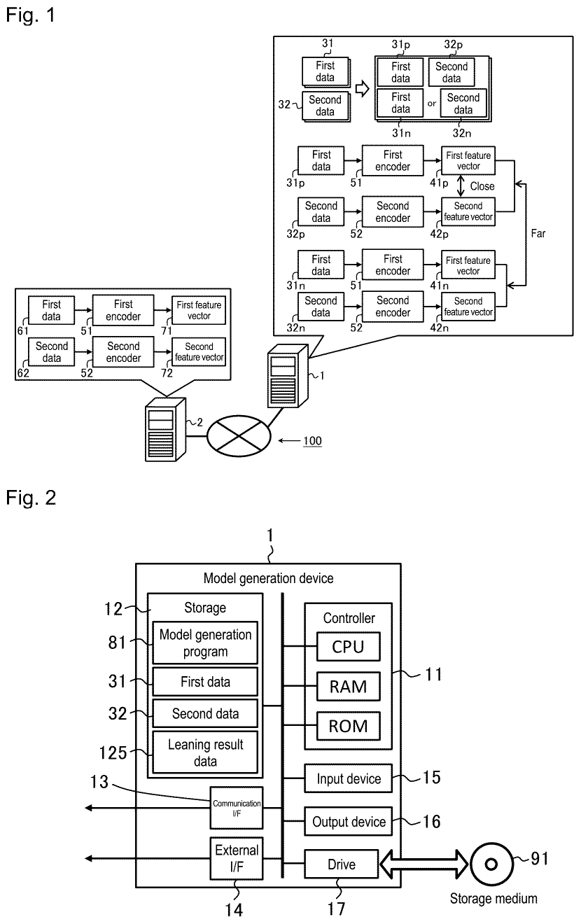

FIG. 1 schematically illustrates an example of a scene to which the present invention is applied. As illustrated in FIG. 1, an information processing system 100 according to the present embodiment includes a model generation device 1 and a data processing device 2.

The model generation device 1 according to the present embodiment is at least one computer configured to generate a trained machine learning model. Specifically, the model generation device 1 acquires first data 31 and second data 32 regarding a crystal structure of a material. The second data 32 indicates a property of the material with an index different from that of the first data 31. As an example, the first data 31 may indicate information regarding a local structure of the crystal of the material. The second data 32 may indicate information regarding the periodicity of the crystal structure of the material.

The acquired first data 31 and second data 32 include a positive sample and a negative sample. The positive sample includes a combination of first data 31p and second data 32p for the same material. The negative sample includes at least one of first data 31n and second data 32n for a material different from the material of the positive sample.

The model generation device 1 performs the machine learning for a first encoder 51 and a second encoder 52 by using the acquired first data 31 and second data 32. The first encoder 51 is a machine learning model configured to convert first data into a first feature vector. The second encoder 52 is a machine learning model configured to convert second data into a second feature vector. The dimension of the first feature vector is the same as the dimension of the second feature vector.

The machine learning for the first encoder 51 and the second encoder 52 is configured by training the first encoder 51 and the second encoder 52 so that values of a first feature vector 41p and a second feature vector 42p calculated from the first data 31p and the second data 32p of the positive sample are positioned close to each other, and at least one value of a first feature vector 41n and a second feature vector 42n calculated from at least one of the first data 31n and the second data 32n of the negative sample is positioned far from at least one value of the first feature vector 41p and the second feature vector 42p calculated from the positive sample. As a result of this machine learning, a trained first encoder 51 and a trained second encoder 52 are generated.

On the other hand, the data processing device 2 according to the present embodiment is at least one computer configured to execute data processing by using the trained machine learning model generated by the model generation device 1. The data processing device 2 may be referred to as, for example, a data presentation device, a data generation device, an estimation device, or the like according to the content of information processing to be executed. FIG. 1 schematically illustrates an example of a scene in which the data processing device 2 operates as a data presentation device.

Specifically, the data processing device 2 acquires at least one of first data 61 and second data 62 regarding the crystal structure of each of a plurality of target materials. By using at least one of the trained first encoder 51 and the trained second encoder 52, the data processing device 2 converts at least one of the acquired first data 61 and second data 62 for each target material into at least one of a first feature vector 71 and a second feature vector 72. The data processing device 2 maps each value of at least one of the obtained first feature vector 71 and second feature vector 72 of each target material onto a space. Then, the data processing device 2 outputs each value of at least one of the first feature vector 71 and the second feature vector 72 of each target material, each value having been mapped onto the space.

As described above, in the present embodiment, a positive sample and a negative sample to be used for machine learning can be prepared depending on whether or not the materials are the same. Therefore, the model generation device 1 can generate the trained first encoder 51 and the trained second encoder 52 at low cost. In addition, by the machine learning, the trained first encoder 51 and the trained second encoder 52 can acquire the ability to map the first data and the second data for materials with similar features to the proximity range on a feature space. As a result, the data processing device 2 can obtain new knowledge related to the material by using at least one of the generated trained first encoder 51 and trained second encoder 52.

In one example, as illustrated in FIG. 1, the model generation device 1 and the data processing device 2 may be connected to each other via a network. The type of the network may be selected as appropriate from, for example, the Internet, a wireless communication network, a mobile communication network, a telephone network, a dedicated network, and the like. However, the method of exchanging data between the model generation device 1 and the data processing device 2 is not limited to such an example, and may be selected as appropriate according to the embodiment. In another example, data may be exchanged between the model generation device 1 and the data processing device 2 by using a storage medium.

In the example of FIG. 1, the model generation device 1 and the data processing device 2 are separate computers. However, the configuration of the information processing system 100 according to the present embodiment is not limited to such an example, and may be determined as appropriate according to the embodiment. In another example, the model generation device 1 and the data processing device 2 may be an integrated computer. In still another example, at least one of the model generation device 1 and the data processing device 2 may be configured by a plurality of computers.

### § 2 Configuration Example

### [Hardware Configuration]

### <Model Generation Device>

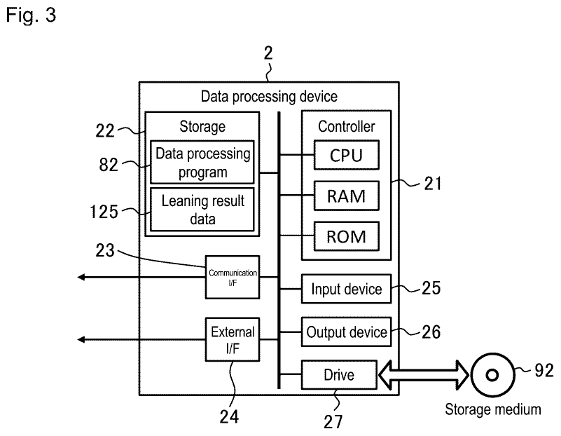

FIG. 2 schematically illustrates an example of the hardware configuration of the model generation device 1 according to the present embodiment. As illustrated in FIG. 2, the model generation device 1 according to the present embodiment is a computer to which a controller 11, a storage 12, a communication interface 13, an external interface 14, an input device 15, an output device 16, and a drive 17 are electrically connected. In FIG. 2, the communication interface and the external interface are referred to as a “communication I/F” and an “external I/F”, respectively. A similar notation is used in FIG. 3 to be described later.

The controller 11 includes a central processing unit (CPU) that is a hardware processor, a random-access memory (RAM), a read-only memory (ROM), and the like, and is configured to execute information processing based on a program and various types of data. The controller 11 (CPU) is an example of a processor resource. The storage 12 is an example of a memory resource, and includes, for example, a hard disk drive, a solid-state drive, or the like. In the present embodiment, the storage 12 stores various types of information such as a model generation program 81, the first data 31, the second data 32, and a learning result data 125.

The model generation program 81 is a program for causing the model generation device 1 to execute information processing (FIG. 8 to be described later) for generating a trained machine learning model. The model generation program 81 includes a series of commands for the information processing. The first data 31 and the second data 32 are used for machine learning. The learning result data 125 indicates information regarding a trained machine learning model generated by machine learning. In the present embodiment, the learning result data 125 is generated as a result of executing the model generation program 81.

The communication interface 13 is, for example, a wired local area network (LAN) module, a wireless LAN module, or the like, and is an interface for performing wired or wireless communication via a network. The model generation device 1 may perform data communication with another computer via the communication interface 13.

The external interface 14 is, for example, a universal serial bus (USB) port, a dedicated port, or the like, and is an interface for connecting to an external device. Any type and number of the external interfaces 14 may be selected. The model generation device 1 may be connected to a device for obtaining each piece of data (31, 32) via the communication interface 13 or the external interface 14.

The input device 15 is, for example, a device for performing input, such as a mouse and a keyboard. The output device 16 is, for example, a device for performing output, such as a display and a speaker. An operator can operate the model generation device 1 by using the input device 15 and the output device 16. The input device 15 and the output device 16 may be integrally configured by, for example, a touch panel display or the like.

The drive 17 is, for example, a compact disc (CD) drive, a digital versatile disc (DVD) drive, or the like, and is a drive device for reading various types of information such as a program stored in a storage medium 91. At least one of the model generation program 81, the first data 31, and the second data 32 may be stored in the storage medium 91.

The storage medium 91 is a medium that accumulates information such as a stored program by electrical, magnetic, optical, mechanical, or chemical action so that a computer, other devices, a machine, or the like can read various types of information such as the program. The model generation device 1 may acquire at least one of the model generation program 81, the first data 31, and the second data 32 from the storage medium 91.

Here, FIG. 2 illustrates a disc-type storage medium such as a CD or a DVD as an example of the storage medium 91. However, the type of the storage medium 91 is not limited to the disc type, and may be other than the disc type. Examples of the storage medium other than the disc type include a semiconductor memory such as a flash memory. The type of the drive 17 may be selected as appropriate according to the type of the storage medium 91.

Regarding a specific hardware configuration of the model generation device 1, components can be omitted, replaced, and added as appropriate according to the embodiment. For example, the controller 11 may include a plurality of hardware processors. The hardware processor may include a microprocessor, a field-programmable gate array (FPGA), a digital signal processor (DSP), or the like. The storage 12 may include the RAM and the ROM included in the controller 11. At least one of the communication interface 13, the external interface 14, the input device 15, the output device 16, and the drive 17 may be omitted. The model generation device 1 may include a plurality of computers. In this case, the hardware configurations of the respective computers may or may not be the same. In addition, the model generation device 1 may be a general-purpose server device, a general-purpose personal computer (PC), or the like, in addition to an information processing device designed exclusively for a service to be provided.

### <Data Processing Device>

FIG. 3 schematically illustrates an example of the hardware configuration of the data processing device 2 according to the present embodiment. As illustrated in FIG. 3, the data processing device 2 according to the present embodiment is a computer to which a controller 21, a storage 22, a communication interface 23, an external interface 24, an input device 25, an output device 26, and a drive 27 are electrically connected.

The controller 21 to the drive 27 and a storage medium 92 of the data processing device 2 may be configured in the same manner as the controller 11 to the drive 17 and the storage medium 91 of the model generation device 1, respectively. The controller 21 includes a CPU, a RAM, a ROM, and the like, which are hardware processors, and is configured to execute various types of information processing, based on a program and data. The controller 21 (CPU) is an example of a processor resource. The storage 22 is an example of a memory resource, and includes, for example, a hard disk drive, a solid-state drive, or the like. In the present embodiment, the storage 22 stores various types of information such as a data processing program 82 and the learning result data 125.

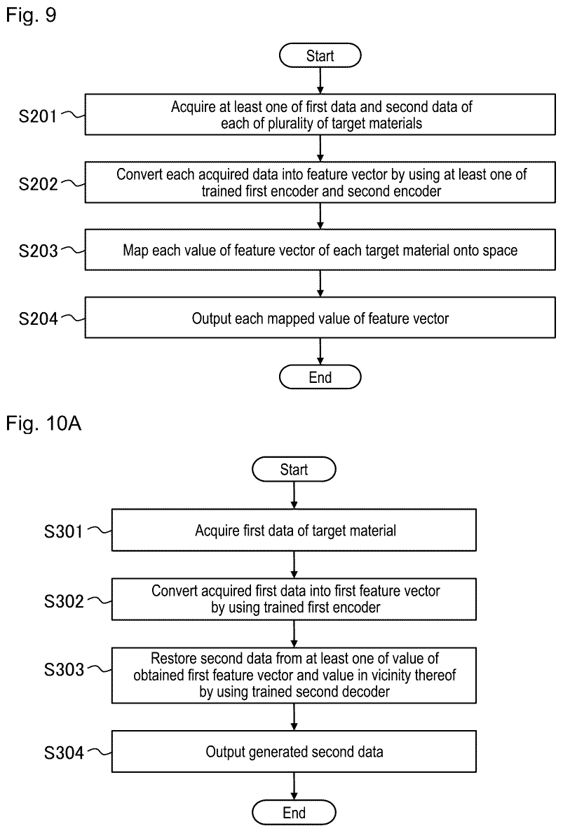

The data processing program 82 is a program for causing the data processing device 2 to execute information processing (FIGS. 9 to 11 to be described later) on data regarding a crystal structure of a target material, using a trained machine learning model. The data processing program 82 includes a series of commands of the information processing. At least one of the data processing program 82 and the learning result data 125 may be stored in the storage medium 92. The data processing device 2 may acquire at least one of the data processing program 82 and the learning result data 125 from the storage medium 92.

The data processing device 2 may perform data communication with another computer via the communication interface 23. The data processing device 2 may be connected to a device for obtaining the first data or the second data via the communication interface 23 or the external interface 24. The data processing device 2 may receive an operation and an input from the operator by using the input device 25 and the output device 26.

Regarding a specific hardware configuration of the data processing device 2, components can be omitted, replaced, and added as appropriate according to the embodiment. For example, the controller 21 may include a plurality of hardware processors. The hardware processor may be configured by a microprocessor, an FPGA, a DSP, or the like. The storage 22 may include the RAM and the ROM included in the controller 21. At least one of the communication interface 23, the external interface 24, the input device 25, the output device 26, and the drive 27 may be omitted. The data processing device 2 may include a plurality of computers. In this case, the hardware configurations of the respective computers may or may not be the same. Further, the data processing device 2 may be a general-purpose server device, a general-purpose PC, or the like, in addition to an information processing device designed exclusively for a service to be provided.

### [Software Configuration]

### <Model Generation Device>

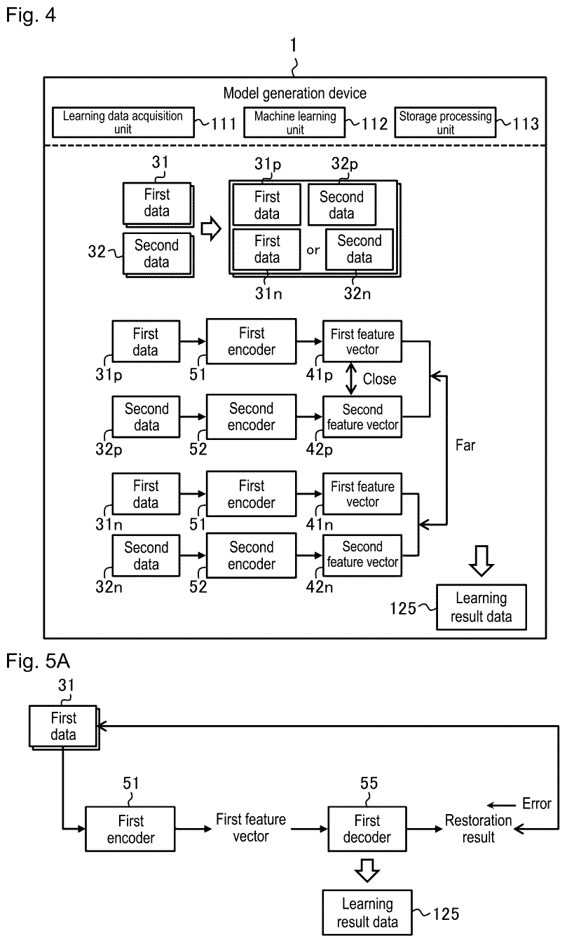

FIG. 4 schematically illustrates an example of the software configuration of the model generation device 1 according to the present embodiment. The controller 11 of the model generation device 1 loads the model generation program 81 stored in the storage 12 in the RAM. Then, the controller 11 causes the CPU to execute a command included in the model generation program 81 loaded in the RAM. As a result, as illustrated in FIG. 4, the model generation device 1 according to the present embodiment operates as a computer that includes a learning data acquisition unit 111, a machine learning unit 112, and a storage processing unit 113 as software modules. That is, in the present embodiment, each software module of the model generation device 1 is achieved by the controller 11 (CPU).

The learning data acquisition unit 111 is configured to acquire the first data 31 and the second data 32 for learning. The first data 31 and the second data 32 relate to the crystal structure of the material, and indicate the properties of the material by indices different from each other. The acquired first data 31 and second data 32 include a plurality of positive samples and a plurality of negative samples. Each position sample includes a combination of the first data 31p and the second data 32p for the same material. Each negative sample includes at least one of the first data 31n and the second data 32n for a material different from the material of the corresponding positive sample (any of the plurality of positive samples).

The machine learning unit 112 is configured to perform machine learning for the first encoder 51 and the second encoder 52 by using the acquired first data 31 and second data 32. The first encoder 51 is configured to convert the first data into a first feature vector. The second encoder 52 is configured to convert the second data into a second feature vector having the same dimension as the dimension of the first feature vector. That is, the encoders (51, 52) are configured to map the first data and the second data, respectively, onto the same dimensional feature space.

The machine learning for the first encoder 51 and the second encoder 52 is configured by training the first encoder 51 and the second encoder 52 so that values of the first feature vector 41p and the second feature vector 42p calculated from the first data 31p and the second data 32p of each positive sample are positioned close to each other, and at least one value of the first feature vector 41n and the second feature vector 42n calculated from at least one of the first data 31n and the second data 32n of each negative sample is positioned far from at least one value of the first feature vector 41p and the second feature vector 42p calculated from the corresponding positive sample.

That is, in the machine learning, the first encoder 51 and the second encoder 52 are trained so that a first distance between the feature vectors (41p, 42p) of the respective positive samples becomes relatively shorter than a second distance between the feature vectors of the corresponding negative samples. This training may include at least one of adjustment for reducing the first distance and adjustment for increasing the second distance. Note that the second distance may be configured by at least one of a distance between the first feature vectors (41p, 41n), a distance between the first feature vector 41p and the second feature vector 42n, a distance between the second feature vector 42p and the first feature vector 41n, and a distance between the second feature vectors (42p, 42n) of the corresponding positive samples and negative samples. The first feature vector (41p, 41n) is calculated from the first data (31p, 31n) using the first encoder 51. The second feature vector (42p, 42n) is calculated from the second data (32p, 32n) using the second encoder 52. As a result of the machine learning, a trained first encoder 51 and a trained second encoder 52 are generated.

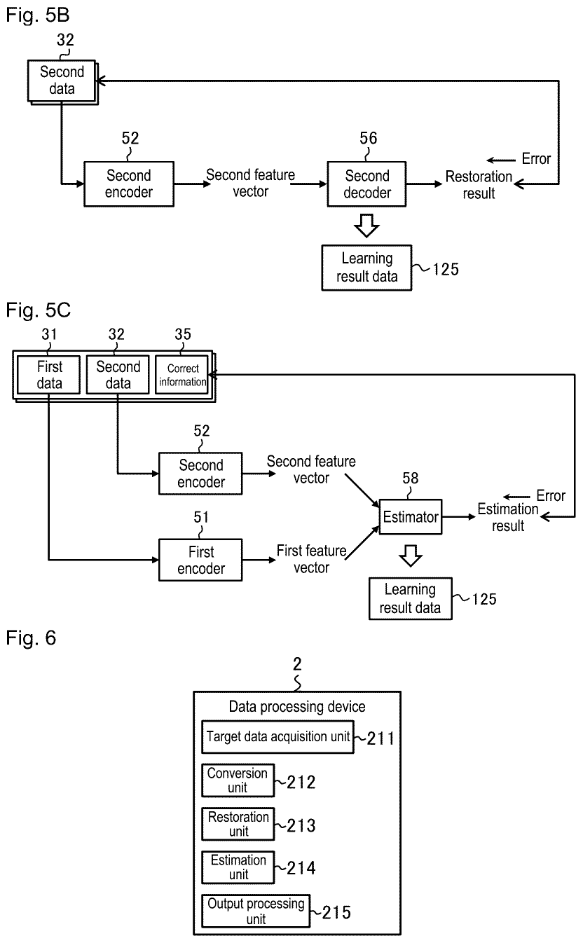

Further, as illustrated in FIGS. 5A to 5C, the model generation device 1 according to the present embodiment may be configured to further generate at least one of a trained first decoder 55, a trained second decoder 56, and a trained estimator 58. The first decoder 55 corresponds to the first encoder 51, and is configured to restore the first data from the first feature vector. The second decoder 56 corresponds to the second encoder 52, and is configured to restore the second data from the second feature vector. The estimator 58 is configured to estimate a characteristic of a material from at least one of a first feature vector and a second feature vector.

FIG. 5A schematically illustrates an example of the course of machine learning for the first decoder 55 by the model generation device 1 according to the present embodiment. When the model generation device 1 is configured to generate the trained first decoder 55, the machine learning unit 112 may be configured to further perform machine learning for the first decoder 55 by using the first data 31. The machine learning for the first decoder 55 is configured by training the first decoder 55 so that a result of restoring the first data 31 by the first decoder 55 from a first feature vector, calculated from the first data 31 by using the first encoder 51, matches the first data 31. As a result of this machine learning, the trained first decoder 55 can be generated.

FIG. 5B schematically illustrates an example of the course of machine learning for the second decoder 56 by the model generation device 1 according to the present embodiment. When the model generation device 1 is configured to generate the trained second decoder 56, the machine learning unit 112 may be configured to further perform machine learning for the second decoder 56 by using the second data 32. The machine learning for the second decoder 56 is configured by training the second decoder 56 so that a result of restoring the second data 32 by the second decoder 56 from the second feature vector, calculated from the second data 32 by using the second encoder 52, matches the second data 32. As a result of this machine learning, the trained second decoder 56 can be generated.

FIG. 5C schematically illustrates an example of the course of machine learning for the estimator 58 by the model generation device 1 according to the present embodiment. When the model generation device 1 is configured to generate the trained estimator 58, the learning data acquisition unit 111 may be configured to further acquire correct information (correct label) 35 indicating the characteristic (true values) of the material. The machine learning unit 112 may be configured to further perform machine learning for the estimator 58 by using the correct information 35 and at least one of the first data 31 and the second data 32. The machine learning for the estimator 58 is configured by training the estimator 58 so that a result of the estimator 58 estimating the characteristic of the material from at least one of the first feature vector, calculated from the first data 31 by using the first encoder 51, and the second feature vector, calculated from the second data 32 by using the second encoder 52, matches the corresponding correct information 35. As a result of this machine learning, the trained estimator 58 can be generated.

As illustrated in FIGS. 4 and 5A to 5C, the storage processing unit 113 is configured to generate, as learning result data 125, information regarding a trained machine learning model (in the present embodiment, the first encoder 51, the second encoder 52, the first decoder 55, the second decoder 56, and the estimator 58) generated by machine learning, and store the generated learning result data 125 in any suitable storage area. The learning result data 125 may be appropriately configured to include information for reproducing the trained machine learning model.

### (Example of Machine Learning Model)

In the present embodiment, the first encoder 51, the second encoder 52, the first decoder 55, the second decoder 56, and the estimator 58 are configured by a machine learning model including one or more arithmetic parameters used for each calculation. As long as each of the above calculations can be executed, the type and structure of the machine learning model to be adopted for each calculation are not particularly limited, and may be selected as appropriate according to the embodiment. As an example, each of the first encoder 51, the second encoder 52, the first decoder 55, and the second decoder 56 may be configured by a neural network or the like. The estimator 58 may be configured by a neural network, a support vector machine, a regression model, a decision tree model, or the like.

The training is configured by adjusting (optimizing) values of the arithmetic parameters to derive an output matching the training data (first data 31/second data 32) from the training data. The machine learning method may be selected as appropriate according to the type of the machine learning model to be adopted. As an example, a method such as backpropagation, solving an optimization problem, or executing regression analysis may be adopted as the machine learning method.

In a case where the neural network is adopted, typically, each of the first encoder 51, the second encoder 52, the first decoder 55, the second decoder 56, and the estimator 58 is configured to include an input layer, one or more intermediate layers (hidden layers), and an output layer. For each layer, for example, any type of layer such as a fully connected layer may be adopted. The number of layers included in each of the above components, the type of each layer, the number of nodes (neurons) of each layer, and the connection relationship of the nodes may be determined as appropriate according to the embodiment. The weight of the connection between the nodes, the threshold value of each node, and the like are examples of the above arithmetic parameters. An example of a training process when a neural network is adopted for each of the first encoder 51, the second encoder 52, the first decoder 55, the second decoder 56, and the estimator 58 will be described below.

### (A) Training of Encoder

As illustrated in FIG. 4, as an example of the training process when each encoder (51, 52) is configured using a neural network, the machine learning unit 112 inputs the first data 31p of each positive sample to the first encoder 51 and executes forward propagation arithmetic processing for the first encoder 51. As a result of this arithmetic processing, the machine learning unit 112 acquires the first feature vector 41p corresponding to the first data 31p of each positive sample from the first encoder 51. Similarly, the machine learning unit 112 inputs the second data 32p of each positive sample to the second encoder 52 and executes forward propagation arithmetic processing for the second encoder 52. As a result of this arithmetic processing, the machine learning unit 112 acquires the second feature vector 42p corresponding to the second data 32p of each positive sample from the second encoder 52.

Further, when the first data 31n is included in the negative sample corresponding to each positive sample, the machine learning unit 112 inputs the first data 31n of the corresponding negative sample to the first encoder 51 and executes forward propagation arithmetic processing for the first encoder 51. As a result of this arithmetic processing, the machine learning unit 112 acquires the first feature vector 41n corresponding to the first data 31n from the first encoder 51. Similarly, when the second data 32n is included in the negative sample corresponding to each positive sample, the machine learning unit 112 inputs the second data 32n of the corresponding negative sample to the second encoder 52 and executes forward propagation arithmetic processing for the second encoder 52. As a result of this arithmetic processing, the machine learning unit 112 acquires the second feature vector 42n corresponding to the second data 32n from the second encoder 52.

The machine learning unit 112 calculates an error from the calculated values of the respective feature vectors to achieve at least one of an operation of decreasing the first distance (bringing the vector values of the positive sample closer to each other) and an operation of increasing the second distance (moving the vector values of the positive sample and the negative sample away from each other). Any loss function may be used to calculate the error as long as at least one of the operation of decreasing the first distance and the operation of increasing the second distance can be achieved. Examples of the loss function capable of achieving the operation include Triplet Loss, Contrastive Loss, Lifted Structure Loss, N-Pair Loss, Angular Loss, and Divergence Loss.

The machine learning unit 112 calculates the gradient of the calculated error. Next, the machine learning unit 112 back-propagates the calculated error gradient by backpropagation to calculate errors in the values of the arithmetic parameters of the first encoder 51 and the second encoder 52. Then, the machine learning unit 112 updates the values of the arithmetic parameters, based on the calculated error.

Through this series of the update process, the machine learning unit 112 adjusts the values of the arithmetic parameters of the first encoder 51 and the second encoder 52 so that the first distance between the feature vectors (41p, 42p) of the respective positive samples becomes shorter than the second distance between the feature vector of the respective positive samples and the feature vector of the corresponding negative sample. The adjustment of the values of the arithmetic parameters may be repeated until a predetermined condition is met, for example, the adjustment is performed a specified number of times or the sum of the calculated errors meets a predetermined index. In addition, conditions for machine learning such as a learning rate may be set as appropriate according to the embodiment. This machine learning process enables the generation of the trained first encoder 51 and the trained second encoder 52 that have acquired the ability to map the first data and the second data for the same material to close positions on the feature space and to map the first data and the second data for different materials to far positions.

### (B) Training of First Decoder

As illustrated in FIG. 5A, as an example of the training process when the first decoder 55 is configured using a neural network, the machine learning unit 112 inputs each first data 31 to the first encoder 51 and executes forward propagation arithmetic processing for the first encoder 51. As a result of this arithmetic processing, the machine learning unit 112 acquires the first feature vector corresponding to each first data 31 from the first encoder 51. The machine learning unit 112 inputs each obtained first feature vector to the first decoder 55 and executes forward propagation arithmetic processing for the first decoder 55. As a result of this arithmetic processing, the machine learning unit 112 acquires an output value corresponding to the result of restoring the first data 31 from each first feature vector from the first decoder 55.

The machine learning unit 112 calculates an error between the acquired output value and the corresponding first data 31, and further calculates the gradient of the calculated error. The machine learning unit 112 back-propagates the calculated error gradient by backpropagation to calculate an error in the value of the arithmetic parameter of the first decoder 55. Then, the machine learning unit 112 updates the value of the arithmetic parameter of the first decoder 55, based on the calculated error.

Through this series of the update process, the machine learning unit 112 adjusts the value of the arithmetic parameter of the first decoder 55 so that the sum of the errors between the restoration result (output value) and the true value (corresponding first data 31) is reduced for each first data 31. The adjustment of the values of the arithmetic parameters may be repeated until a predetermined condition is met, for example, the adjustment is performed a specified number of times or the sum of the calculated errors is equal to or less than a threshold value. In addition, conditions of machine learning such as a loss function and a learning rate may be set as appropriate according to the embodiment. This machine learning process enables the generation of the trained first decoder 55 that has acquired the ability to restore the corresponding first data from the first feature vector obtained by the first encoder 51.

As long as the trained first decoder 55 that has acquired the ability to restore the first data from the first feature vector can be generated, the timing of executing machine learning for the first decoder 55 is not particularly limited, and may be selected as appropriate according to the embodiment. In one example, the machine learning for the first decoder 55 may be executed after the machine learning for the first encoder 51 and the second encoder 52. In this case, the trained first encoder 51 may be used for the machine learning for the first decoder 55. In another example, the machine learning for the first decoder 55 may be executed simultaneously with the machine learning for the first encoder 51 and the second encoder 52. In this case, the machine learning unit 112 may also back-propagate the gradient of the error in the machine learning for the first decoder 55 to the first encoder 51 to calculate the error of the value of the arithmetic parameter of the first encoder 51. Then, the machine learning unit 112 may update the value of the arithmetic parameter of the first encoder 51 together with the value of the arithmetic parameter of the first decoder 55, based on the calculated error.

### (C) Training of Second Decoder

As illustrated in FIG. 5B, as an example of the training process when the second decoder 56 is configured using a neural network, the machine learning unit 112 inputs each second data 32 to the second encoder 52 and executes forward propagation arithmetic processing for the second encoder 52. As a result of this arithmetic processing, the machine learning unit 112 acquires the second feature vector corresponding to each second data 32 from the second encoder 52. The machine learning unit 112 inputs each obtained second feature vector to the second decoder 56 and executes forward propagation arithmetic processing for the second decoder 56. As a result of this arithmetic processing, the machine learning unit 112 acquires an output value corresponding to the result of restoring the second data 32 from each second feature vector from the second decoder 56.

The machine learning unit 112 calculates an error between the acquired output value and the corresponding second data 32, and further calculates the gradient of the calculated error. The machine learning unit 112 back-propagates the calculated error gradient by backpropagation to calculate an error in the value of the arithmetic parameter of the second decoder 56. Then, the machine learning unit 112 updates the value of the arithmetic parameter of the second decoder 56, based on the calculated error.

Through this series of the update process, the machine learning unit 112 adjusts the value of the arithmetic parameter of the second decoder 56 so that the sum of the errors between the restoration result (output value) and the true value (corresponding second data 32) is reduced for each second data 32. The adjustment of the values of the arithmetic parameters may be repeated until a predetermined condition is met, for example, the adjustment is performed a specified number of times or the sum of the calculated errors is equal to or less than a threshold value. In addition, conditions of machine learning such as a loss function and a learning rate may be set as appropriate according to the embodiment. This machine learning process enables the generation of the trained second decoder 56 that has acquired the ability to restore the corresponding second data from the second feature vector obtained by the second encoder 52.

As long as the trained second decoder 56 that has acquired the ability to restore the second data from the second feature vector can be generated, the timing of executing machine learning for the second decoder 56 is not particularly limited, and may be selected as appropriate according to the embodiment. In one example, the machine learning for the second decoder 56 may be executed after the machine learning for the first encoder 51 and the second encoder 52. In this case, the trained second encoder 52 may be used for the machine learning for the second decoder 56. In another example, the machine learning for the second decoder 56 may be executed simultaneously with the machine learning for the first encoder 51 and the second encoder 52. In this case, the machine learning unit 112 may also back-propagate the gradient of the error in the machine learning for the second decoder 56 to the second encoder 52 to calculate the error of the value of the arithmetic parameter of the second encoder 52. Then, the machine learning unit 112 may update the value of the arithmetic parameter of the second encoder 52 together with the value of the arithmetic parameter of the second decoder 56, based on the calculated error.

Further, in one example, the machine learning for the second decoder 56 may be executed in parallel with the machine learning for the first decoder 55. In another example, the machine learning for the second decoder 56 may be executed separately from the machine learning for the first decoder 55. In this case, the machine learning process may be first executed for either the first decoder 55 or the second decoder 56.

### (D) Training of Estimator

As illustrated in FIG. 5C, a plurality of data sets each configured by a combination of at least one of the first data 31 and the second data 32 with the correct information 35 of the corresponding material are used for the machine learning for the estimator 58. An example of the training process when the estimator 58 is configured using a neural network will be described below.

In a case where the estimator 58 is trained to estimate the characteristic of the material from the first feature vector, the machine learning unit 112 inputs the first data 31 of each data set to the first encoder 51 and executes forward propagation arithmetic processing for the first encoder 51. As a result of this arithmetic processing, the machine learning unit 112 acquires the first feature vector corresponding to each first data 31 from the first encoder 51. The machine learning unit 112 inputs each obtained first feature vector to the estimator 58 and executes forward propagation arithmetic processing for the estimator 58. As a result of this arithmetic processing, the machine learning unit 112 acquires an output value corresponding to the result of estimating the characteristic of each material from the estimator 58.

In a case where the estimator 58 is trained to estimate the characteristic of the material from the second feature vector, the machine learning unit 112 inputs the second data 32 of each data set to the second encoder 52 and executes forward propagation arithmetic processing for the second encoder 52. As a result of this arithmetic processing, the machine learning unit 112 acquires the second feature vector corresponding to each second data 32 from the second encoder 52. The machine learning unit 112 inputs each obtained second feature vector to the estimator 58 and executes forward propagation arithmetic processing for the estimator 58. As a result of this arithmetic processing, the machine learning unit 112 acquires an output value corresponding to the result of estimating the characteristic of each material from the estimator 58.

Note that the estimator 58 may be configured to receive inputs f both the first feature vector and the second feature vector, or may be configured to receive input of only one of the first feature vector and the second feature vector. When the estimator 58 is configured to receive both the first feature vector and the second feature vector, the machine learning unit 112 inputs the first feature vector and the second feature vector derived from the first data 31 and the second data 32 for the same material to the estimator 58, and acquires an output value corresponding to a result of estimating the characteristic of the material from the estimator 58.

Next, the machine learning unit 112 calculates an error between the acquired output value and the true value indicated by the corresponding correct information 35, and further calculates the gradient of the calculated error. The machine learning unit 112 back-propagates the calculated error gradient by backpropagation to calculate an error in the value of the arithmetic parameter of the estimator 58. Then, the machine learning unit 112 updates the value of the arithmetic parameter of the estimator 58, based on the calculated error.

Through this series of the update process, the machine learning unit 112 adjusts the value of the arithmetic parameter of the estimator 58 so that the sum of errors between the output value of the estimation result derived from at least one of the first data 31 and the second data 32 and the true value indicated by the corresponding correct information 35 is reduced for each data set. The adjustment of the values of the arithmetic parameters may be repeated until a predetermined condition is met, for example, the adjustment is performed a specified number of times or the sum of the calculated errors is equal to or less than a threshold value. In addition, conditions of machine learning such as a loss function and a learning rate may be set as appropriate according to the embodiment. This machine learning process enables the generation of the trained estimator 58 that has acquired the ability to estimate the characteristic of the material from at least one of the first feature vector and the second feature vector.

As long as the trained estimator 58 that has acquired the ability to estimate the characteristic of the material can be generated, the timing of executing machine learning for the estimator 58 is not particularly limited, and may be selected as appropriate according to the embodiment. In one example, the machine learning for the estimator 58 may be executed after the machine learning for the first encoder 51 and the second encoder 52. In this case, when training is performed to estimate the characteristic of the material from the first feature vector, the trained first encoder 51 may be used for the machine learning for the estimator 58. When training is performed to estimate the characteristic of the material from the second feature vector, the trained second encoder 52 may be used for the machine learning for the estimator 58. In another example, the machine learning for the estimator 58 may be executed simultaneously with the machine learning for the first encoder 51 and the second encoder 52. In this case, when training is performed to estimate the characteristic of the material from the first feature vector, the machine learning unit 112 may also back-propagate the gradient of the error in the machine learning for the estimator 58 to the first encoder 51 to calculate the error of the value of the arithmetic parameter of the first encoder 51. Then, the machine learning unit 112 may update the value of the arithmetic parameter of the first encoder 51 together with the value of the arithmetic parameter of the estimator 58, based on the calculated error. Further, when training is performed to estimate the characteristic of the material from the second feature vector, the machine learning unit 112 may also back-propagate the gradient of the error in the machine learning for the estimator 58 to the second encoder 52 to calculate the error of the value of the arithmetic parameter of the second encoder 52. Then, the machine learning unit 112 may update the value of the arithmetic parameter of the second encoder 52 together with the value of the arithmetic parameter of the estimator 58, based on the calculated error.

Further, in one example, the machine learning for the estimator 58 may be executed simultaneously with at least one of the machine learning for the first decoder 55 and the machine learning for the second decoder 56. In another example, the machine learning for the estimator 58 may be executed separately from the machine learning for the first decoder 55 and the second decoder 56. In this case, the machine learning may be first executed for either the estimator 58 or each decoder (55, 56).

Further, in another example, the estimator 58 may include a machine learning model other than a neural network, such as a support vector machine or a regression model. In this case as well, the machine learning for the estimator 58 is configured by adjusting the value of the arithmetic parameter of the estimator 58 so that the output value of the estimation result derived from at least one of the first data 31 and the second data 32 approaches (e.g., coincides with) the true value indicated by the corresponding correct information 35 for each data set. The method of adjusting the value of the arithmetic parameter of the estimator 58 may be selected as appropriate according to the machine learning model to be adopted. As an example, a method such as solving an optimization problem or performing a regression analysis may be adopted as the method of adjusting the value of the arithmetic parameter of the estimator 58.

### (Storage Process)

The storage processing unit 113 stores, as the learning result data 125, the trained machine learning model (first encoder 51, second encoder 52, first decoder 55, second decoder 56, and estimator 58) generated by each machine learning described above. The configuration of the learning result data 125 is not particularly limited as long as the information for executing the calculation of the trained machine learning model can be held, and may be determined as appropriate according to the embodiment. As an example, the learning result data 125 may be configured to include a configuration of a machine learning model (e.g., neural network structure, etc.) and information indicating a value of an arithmetic parameter adjusted by the above machine learning. The learning result data 125 may be stored in any storage area. The learning result data 125 may be referred to as appropriate to set the trained machine learning model to be usable on the computer.

In the example of FIGS. 4 and 5A to 5C, for convenience of description, information regarding all of the first encoder 51, the second encoder 52, the first decoder 55, the second decoder 56, and the estimator 58 is included in the learning result data 125. However, the form of holding the learning results is not limited to such an example. Information regarding at least one of the first encoder 51, the second encoder 52, the first decoder 55, the second decoder 56, and the estimator 58 may be held as separate learning result data. In another example, independent learning result data may be generated for each of the first encoder 51, the second encoder 52, the first decoder 55, the second decoder 56, and the estimator 58.

### <Data Processing Device>

FIG. 6 schematically illustrates an example of the software configuration of the data processing device 2 according to the present embodiment. The controller 21 of the data processing device 2 loads the data processing program 82 stored in the storage 22 in the RAM. Then, the controller 21 causes the CPU to execute a command included in the data processing program 82 loaded in the RAM. As a result, as illustrated in FIG. 6, the data processing device 2 according to the present embodiment operates as a computer including a target data acquisition unit 211, a conversion unit 212, a restoration unit 213, an estimation unit 214, and an output processing unit 215 as software modules. That is, in the present embodiment, the controller 21 (CPU) achieves each software module of the data processing device 2 in the same manner as the model generation device 1.

By including at least one of the trained first encoder 51 and the trained second encoder 52 generated by the model generation device 1, it is possible to configure a data presentation device that presents a value of a feature vector calculated from at least one of the first data and the second data. By including the trained first encoder 51 and the trained second decoder 56, it is possible to configure a data generation device that generates the second data from the first data. By including the trained second encoder 52 and the trained first decoder 55, it is possible to configure a data generation device that generates the first data from the second data. By including at least one of the trained first encoder 51 and the trained second encoder 52 and the trained estimator 58, it is possible to configure an estimation device that estimates a characteristic of a material from at least one of the first data and the second data. FIG. 6 illustrates an example of a case where the data processing device 2 is configured to be able to execute the operations of all the devices.

### (A) Data Presentation Device

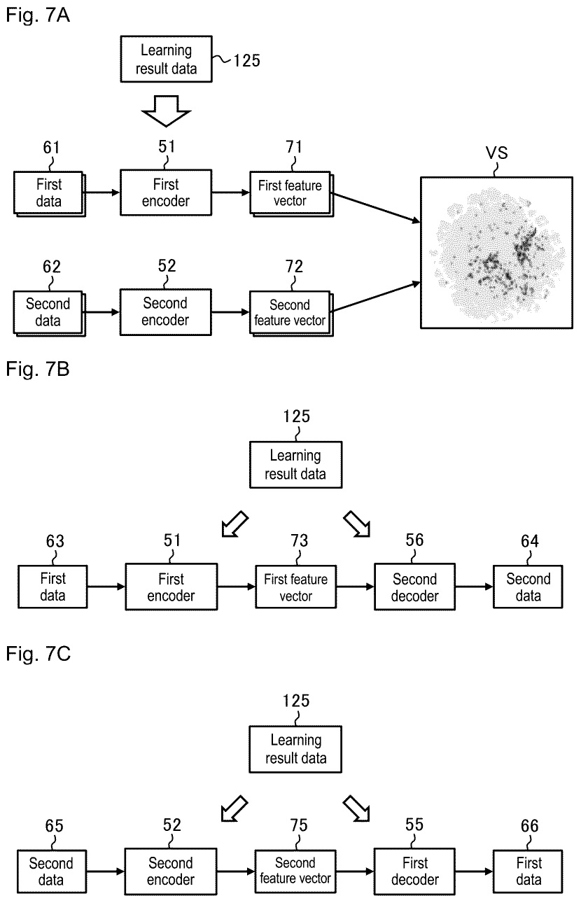

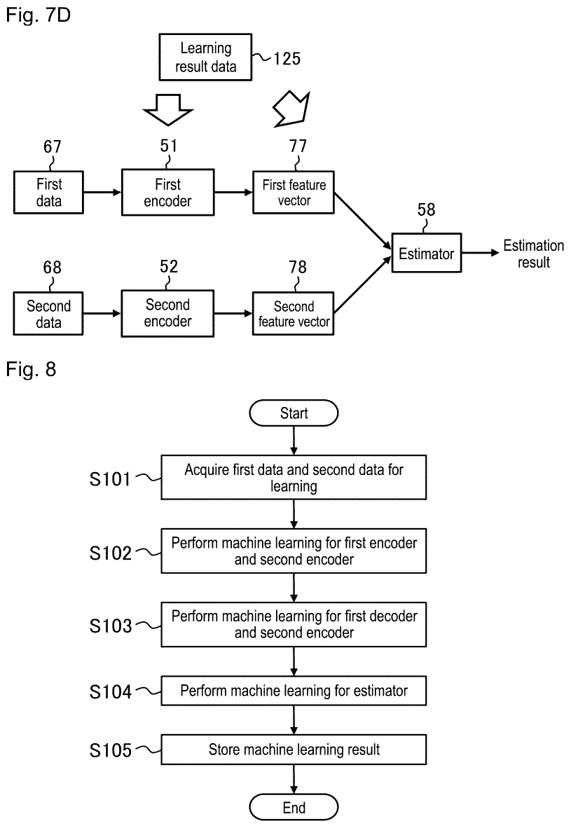

FIG. 7A schematically illustrates an example of the course of the above data presentation process (i.e., a scene in which the data processing device 2 operates as a data presentation device).

In this case, the target data acquisition unit 211 is configured to acquire at least one of the first data 61 and the second data 62 regarding the crystal structure of each of the plurality of target materials. The conversion unit 212 includes at least one of the trained first encoder 51 and the trained second encoder 52 by holding the learning result data 125. The conversion unit 212 is configured to acquire at least one of the first feature vector 71 and the second feature vector 72 by performing at least one of a process of converting the first data 61 for each target material acquired using the trained first encoder 51 into the first feature vector 71 and a process of converting the second data 62 for each target material acquired using the trained second encoder 52 into the second feature vector 72.

The output processing unit 215 is configured to map each value of at least one of the obtained first feature vector 71 and second feature vector 72 of each target material onto a space VS, and output each value of at least one of the first feature vector 71 and second feature vector 72 of each target material, each value having been mapped onto the space VS. In one example, the output processing unit 215 may be configured to directly map each value of at least one of the obtained first feature vector 71 and second feature vector 72 of each target material onto the space VS. In another example, the output processing unit 215 may be configured to convert each value of at least one of the first feature vector 71 and the second feature vector 72 of each obtained target material to a dimension lower than the original dimension to maintain the positional relationship of each of the values in the mapping process, and then map each converted value onto the space VS. In this case, the output processing unit 215 may be configured to output each converted value of at least one of the first feature vector 71 and the second feature vector 72 of each target material in the process of outputting each value. As a result, it is possible to improve the efficiency of the output resources (e.g., saving space in an information output range, improving visibility, etc.) while reducing the impact on the information regarding the similarity of each target material.

Note that the data processing device 2 may be configured to present both the first feature vector 71 and the second feature vector 72 in the space VS. Alternatively, the data processing device 2 may be configured to present only one of the first feature vector 71 and the second feature vector 72 in the space VS.

(B) Data Generation Device for Generating Second Data from First Data

FIG. 7B schematically illustrates an example of the course of a process to generate second data 64 from first data 63 (i.e., a scene where the data processing device 2 operates as a data generation device that generates the second data from the first data).

In this case, the target data acquisition unit 211 is configured to acquire the first data 63 for the target material. The conversion unit 212 includes the trained first encoder 51 by holding the learning result data 125. The conversion unit 212 is configured to convert the acquired first data 63 for the target material into a first feature vector 73 by using the trained first encoder 51. The restoration unit 213 includes the trained second decoder 56 by holding the learning result data 125. The restoration unit 213 is configured to restore the second data 64 from at least one of the value of the first feature vector 73 obtained by the conversion and the value in the proximity thereof by using the trained second decoder 56 to generate the second data 64. The output processing unit 215 is configured to output the generated second data 64.

(C) Data Generation Device for Generating First Data from Second Data

FIG. 7C schematically illustrates an example of the course of a process to generate first data 66 from second data 65 (i.e., a scene where the data processing device 2 operates as a data generation device that generates the first data from the second data).

In this case, the target data acquisition unit 211 is configured to acquire the second data 65 for the target material. The conversion unit 212 includes the trained second encoder 52 by holding the learning result data 125. The conversion unit 212 is configured to convert the acquired second data 65 for the target material into a second feature vector 75 by using the trained second encoder 52. The restoration unit 213 includes a trained first decoder 55 by holding the learning result data 125. The restoration unit 213 is configured to restore the first data 66 from at least one of the value of the second feature vector 75 obtained by the conversion and the value in the proximity thereof by using the trained first decoder 55 to generate the first data 66. The output processing unit 215 is configured to output the generated first data 66.

### (D) Estimation Device

FIG. 7D schematically illustrates an example of the course of a process to estimate the characteristic of the target material from at least one of the first feature vector and the second feature vector (i.e., the data processing device 2 operates as an estimation device).

In this case, the target data acquisition unit 211 is configured to acquire at least one of first data 67 and second data 68 regarding the crystal structure of the target material. The conversion unit 212 includes at least one of the trained first encoder 51 and the trained second encoder 52 by holding the learning result data 125. When the data processing device 2 is configured to estimate the characteristic of the target material from the first feature vector, the conversion unit 212 is configured to include the trained first encoder 51. When the data processing device 2 is configured to estimate the characteristic of the target material from the second feature vector, the conversion unit 212 is configured to include the trained second encoder 52. The conversion unit 212 is configured to convert at least one of the acquired first data 67 and second data 68 into at least one of a first feature vector 77 and a second feature vector 78 by using at least one of the trained first encoder 51 and the trained second encoder 52. The estimation unit 214 includes the trained estimator 58 by holding the learning result data 125. The estimation unit 214 is configured to estimate the characteristic of the target material from at least one value of the obtained first feature vector 77 and second feature vector 78 using the trained estimator 58. The output processing unit 215 is configured to output a result of estimating the characteristic of the target material.

### <Each Data>

The first data (31, 61, 63, 66, 67) and the second data (32, 62, 64, 65, 68) are configured to indicate information regarding the crystal structure of the material. The first data 31 and the second data 32 are used for machine learning and relate to a material for learning. The first data (61, 63, 67) and the second data (62, 65, 68) are used for each inference process such as data presentation and relate to a material (target material) that is a target of each inference process. The material is a substance with a structure in which atoms or molecules are arranged (thereby expressing a function). As long as the first data and the second data can be acquired, it does not matter whether the material is actually present or a virtual substance on a computer. The first data (31, 61, 63, 67) and the second data (32, 62, 65, 68) may be obtained by actual measurement or may be obtained by simulation.

The first data (31, 61, 63, 66, 67) and the second data (32, 62, 64, 65, 68) indicate the properties of the material with different indices. Each type may be selected as appropriate according to the embodiment. As an example, the first data (31, 61, 63, 66, 67) may indicate the property of the material based on a local perspective of the crystal structure. As a specific example, the first data (31, 61, 63, 66, 67) may indicate information regarding a local structure of the crystal of the material. The second data (32, 62, 64, 65, 68) may indicate the property of the material based on an overall overhead perspective. As a specific example, the second data (32, 62, 64, 65, 68) may indicate information regarding the periodicity of the crystal structure of the material. The periodicity of the crystal structure may be expressed by the presence or absence of periodicity, the state of periodicity (the state of periodic features indicated by the crystal structure), and the like. The material may have periodicity or may not have periodicity.

As an example of the data indicating the information regarding the local structure, the first data (31, 61, 63, 66, 67) may include at least one of three-dimensional atomic position data, Raman spectroscopy data, nuclear magnetic resonance spectroscopy data, infrared spectroscopy data, mass spectrometry data, and X-ray absorption spectroscopy data. When the first data (31, 61, 63, 66, 67) is configured to include three-dimensional atomic position data, the three-dimensional atomic position data may be configured to express a state (e.g., position, type, etc.) of an atom in the material by at least one of a probability density function, a probability distribution function, and a probability mass function. That is, in the three-dimensional atomic position data, the probability related to the state of the atom such as the probability that the target atom is present at the target position and the probability that the target type atom is included may be indicated by at least one of the probability density function, the probability distribution function, and the probability mass function. With these configurations, it is possible to appropriately prepare the first data indicating the characteristic of the material based on the local perspective of the crystal structure.

In addition, as an example of the data indicating information regarding periodicity, the second data (32, 62, 64, 65, 68) may be configured by at least one of X-ray diffraction data, neutron diffraction data, electron beam diffraction data, and total scattering data. As a result, it is possible to appropriately prepare the second data indicating the property of the material based on the overall overhead perspective.

Each feature vector is a sequence of fixed length (as an example, a length of about several tens to a thousand) generated by each encoder (51, 52) and easily handled by a computer. In many cases, each feature vector is configured so that it is difficult for a human to directly understand its meaning. Basically, one feature vector is generated for the first data and another for the second data for each material.

The range of the characteristic of the material estimated from the feature vector at the time of operation as the estimation device depends on the correct information 35 used for the machine learning. The content and number of characteristics of the material estimated from the feature vector are not particularly limited, and may be determined as appropriate according to the embodiment. The characteristics of the material may be, for example, catalytic characteristics, electron mobility, band gap, thermal conductivity, thermoelectric characteristics, mechanical properties (e.g., Young's modulus, sound speed, etc.), and the like.

### <Others>

Each software module of the model generation device 1 and the data processing device 2 will be described in detail in an operation example to be described later. In the present embodiment, an example in which each software module of the model generation device 1 and the data processing device 2 is achieved by a general-purpose CPU has been described. However, some or all of the software modules may be achieved by one or a plurality of dedicated processors. That is, each of the above modules may be achieved as a hardware module. With respect to the software configuration of each of the model generation device 1 and the data processing device 2, software modules may be omitted, replaced, or added as appropriate according to the embodiment.

### § 3 Operation Example

### [Model Generation Device]

FIG. 8 is a flowchart illustrating an example of a process procedure for the model generation device 1 according to the present embodiment. The following process procedure for the model generation device 1 is an example of the model generation method. However, the following process procedure for the model generation device 1 is merely an example, and each step may be changed as much as possible. In addition, with respect to the following process procedure for the model generation device 1, steps can be omitted, replaced, and added as appropriate according to the embodiment.

### (Step S101)

In step S101, the controller 11 operates as the learning data acquisition unit 111, and acquires the first data 31 and the second data 32 for learning that include a plurality of positive samples and a plurality of negative samples. Each position sample includes a combination of the first data 31p and the second data 32p for the same material. Each negative sample includes at least one of the first data 31n and the second data 32n for a material different from the material of the corresponding positive sample.

The first data 31 and the second data 32 may be obtained by actual measurement or may be obtained by simulation. For measurement of each data (31, 32), a measurement device corresponding to each data (31, 32) may be used. The type of the measurement device and the simulation method may be selected as appropriate according to the type of each data (31, 32). As the simulation method, for example, first-principles calculations, molecular dynamics calculations, or the like may be used.

In one example, the controller 11 may directly acquire each of the first data 31 and the second data 32 from the corresponding measurement device. Alternatively, the controller 11 may acquire each of the first data 31 and the second data 32 by executing simulation. In another example, the controller 11 may acquire each of the first data 31 and the second data 32 from a storage area of another computer or an external storage device via, for example, a network, the storage medium 91, or the like. In this case, the first data 31 and the second data 32 may be stored in the same storage area (storage device, storage medium), or may be stored in different storage areas. The number of samples of the first data 31 and the second data 32 to be acquired may be selected as appropriate according to the embodiment.

In the present embodiment, the controller 11 further acquires correct information 35 indicating the characteristic of the material corresponding to at least one of the first data 31 and the second data 32. The correct information 35 may be manually generated or may be generated by any mechanical method. In one example, the correct information 35 may be generated in the model generation device 1. In another example, the controller 11 may acquire the correct information 35 from a storage area of another computer or an external storage device via, for example, a network, the storage medium 91, or the like. Note that the timing of acquiring the correct information 35 is not limited to such an example. The process of acquiring the correct information 35 may be executed at any timing before the machine learning for the estimator 58 is executed in step S104 to be described later.

Upon acquiring the first data 31, the second data 32, and the correct information 35, the controller 11 advances the process to the next step S102.

### (Step S102)

In step S102, the controller 11 operates as the machine learning unit 112, and performs machine learning for the first encoder 51 and the second encoder 52 by using the acquired first data 31 and second data 32. As described above, the controller 11 optimizes the values of the arithmetic parameters of the first encoder 51 and the second encoder 52 by machine learning so that the first distance between the feature vectors of the respective positive samples becomes shorter than the second distance between the feature vector of the respective positive samples and the feature vector of the corresponding negative sample.

The optimization in the machine learning may include at least one of adjustment to decrease the first distance and adjustment to increase the second distance. Further, in this machine learning, the controller 11 may optimize the values of the arithmetic parameters of the first encoder 51 and the second encoder 52 so that the first feature vector 41p and the second feature vector 42p of each positive sample coincide with each other (i.e., the first distance approaches 0).

As a result of the machine learning, it is possible to generate the trained first encoder 51 and the trained second encoder 52 that have acquired the ability to map the first data and the second data for the same material to close positions on the feature space and to map the first data and the second data for different materials to far positions. When the machine learning for the first encoder 51 and the second encoder 52 is completed, the controller 11 advances the process to the next step S103.

### (Step S103)

In step S103, the controller 11 operates as the machine learning unit 112, and performs machine learning for the first decoder 55 by using the first data 31. As described above, the controller 11 optimizes the value of the arithmetic parameter of the first decoder 55 so that the sum of the errors between the output value indicating the restoration result and the corresponding first data 31 is reduced for each first data 31 by machine learning. As a result of this machine learning, it is possible to generate the trained first decoder 55 that has acquired the ability to recover the corresponding first data from the first feature vector obtained by the first encoder 51.

In addition, the controller 11 operates as the machine learning unit 112, and performs machine learning for the second decoder 56 by using the second data 32. As described above, the controller 11 optimizes the value of the arithmetic parameter of the second decoder 56 so that the sum of the errors between the output value indicating the restoration result and the corresponding second data 32 is reduced for each second data 32 by machine learning. As a result of this machine learning, it is possible to generate the trained second decoder 56 that has acquired the ability to recover the corresponding second data from the second feature vector obtained by the second encoder 52. When the machine learning for the first decoder 55 and the second decoder 56 is completed, the controller 11 advances the process to the next step S104.

Note that the timing of executing the machine learning for each of the first decoder 55 and the second decoder 56 is not limited to such an example. In another example, the machine learning for at least one of the first decoder 55 and the second decoder 56 may be executed simultaneously with the machine learning in the above step S102. In a case where the machine learning for the first decoder 55 is executed simultaneously with the machine learning in the above step S102, the controller 11 may also optimize the value of the arithmetic parameter of the first encoder 51, based on the error of the restoration. In a case where the machine learning for the second decoder 56 is executed simultaneously with the machine learning in the above step S102, the controller 11 may also optimize the value of the arithmetic parameter of the second encoder 52, based on the error of the restoration.

Further, the first data 31 used for the machine learning for the first decoder 55 may not completely coincide with the first data (31p, 31n) that can be used for the machine learning for each encoder (51, 52). Similarly, the second data 32 used for the machine learning for the second decoder 56 may not completely coincide with the second data (32p, 32n) that can be used for the machine learning for each encoder (51, 52).

### (Step S104)

In step S104, the controller 11 operates as the machine learning unit 112, and performs machine learning for the estimator 58 by using a plurality of data sets. As described above, by machine learning, the controller 11 optimizes the value of the arithmetic parameter of the estimator 58 so that the sum of errors between the output value of the estimation result derived from at least one of the first data 31 and the second data 32 and the true value indicated by the corresponding correct information 35 is reduced for each data set. As a result of this machine learning, it is possible to generate the trained estimator 58 that has acquired the ability to estimate the characteristic of the material from at least one of the first feature vector and the second feature vector. When the machine learning for the estimator 58 is completed, the controller 11 advances the process to the next step S105.

Note that the timing at which the machine learning for the estimator 58 is executed is not limited to such an example. In another example, the machine learning for the estimator 58 may be executed before the machine learning for at least one of the first decoder 55 and the second decoder 56. Further, in another example, the machine learning for the estimator 58 may be executed simultaneously with the machine learning in the above step S102. In this case, when the estimator 58 is configured to estimate the characteristic of the material from the first feature vector, the controller 11 may also optimize the value of the arithmetic parameter of the first encoder 51, based on the error of the estimation. Similarly, when the estimator 58 is configured to estimate the characteristic of the material from the second feature vector, the controller 11 may also optimize the value of the arithmetic parameter of the second encoder 52, based on the error of the estimation.

In addition, the first data 31 and the second data 32 that can be used for the machine learning for the estimator 58 may not completely coincide with the first data (31p, 31n) and the second data (32p, 32n) that can be used for the machine learning for each encoder (51, 52).

### (Step S105)

In step S105, the controller 11 operates as the storage processing unit 113, and generates, as the learning result data 125, information regarding a trained machine learning model (first encoder 51, second encoder 52, first decoder 55, second decoder 56, and estimator 58) generated by each machine learning. Then, the controller 11 stores the generated learning result data 125 in any suitable storage area.

The storage destination of the learning result data 125 may be, for example, the RAM in the controller 11, the storage 12, an external storage device, a storage medium, or a combination thereof. The storage medium may be, for example, a CD, a DVD, or the like, and the controller 11 may store the learning result data 125 in the storage medium via the drive 17. The external storage device may be, for example, a data server such as a network-attached storage (NAS). In this case, the controller 11 may store the learning result data 125 in the data server via the network using the communication interface 13. Further, the external storage device may be, for example, an externally attached storage device connected to the model generation device 1 via the external interface 14.

When the storage of the learning result data 125 is completed, the controller 11 ends the process procedure for the model generation device 1 according to the present operation example.

Note that the generated learning result data 125 may be provided to the data processing device 2 at any timing. In one example, the controller 11 may transfer the learning result data 125 to the data processing device 2 as the process of the above step S105 or separately from the process of step S105. The data processing device 2 may acquire the learning result data 125 by receiving this transfer. In another example, the data processing device 2 may acquire the learning result data 125 by using the communication interface 23 to access the model generation device 1 or the data server via a network. In another example, the data processing device 2 may acquire the learning result data 125 via the storage medium 92. In another example, the learning result data 125 may be incorporated in the data processing device 2 in advance.

Further, the controller 11 may update or newly create a trained machine learning model by regularly or irregularly repeating the processes of the above steps S101 to S105. In this case, the controller 11 may update or newly create all the machine learning models described above. Alternatively, the controller 11 may update or newly create only some of the machine learning models. At the time of the repetition, at least a part of the first data 31 and the second data 32, which are usable for machine learning, may be subjected to change, correction, addition, deletion, or the like, as appropriate. Then, the controller 11 may provide the updated or newly created learning result data 125 to the data processing device 2 by any method and timing. The learning result data 125 (trained machine learning model) held by the data processing device 2 may be updated in the manner described above.

### [Data Processing Device]

### (A) Data Presentation Process

FIG. 9 is a flowchart illustrating an example of a process procedure related to feature vector presentation by the data processing device 2 according to the present embodiment. The following process procedure related to feature vector presentation is an example of a data presentation method. The command portion of the following process procedure related to feature vector presentation in the data processing program 82 is an example of a data presentation program. However, the following process procedure related to feature vector presentation is merely an example, and each step may be changed as much as possible. In addition, with respect to the following process procedure related to feature vector presentation, steps can be omitted, replaced, and added as appropriate according to the embodiment.

### (Step S201)

In step S201, the controller 21 operates as the target data acquisition unit 211, and acquires at least one of first data 61 and second data 62 regarding the crystal structure of each of the plurality of target materials.

The first data 61 and the second data 62 are of the same type as the first data 31 and the second data 32 for learning. Similarly to the first data 31 and the second data 32, the first data 61 and the second data 62 may be obtained by actual measurement or may be obtained by simulation. In a case where the first data 61 is acquired, at least a part of the acquired first data 61 may overlap with the first data 31 for learning. Similarly, when the second data 62 is acquired, at least a part of the acquired second data 62 may overlap the second data 32 for learning. In one example, at least one of the first data 61 and the second data 62 to be processed may be designated by an operator by any method.

In one example, the controller 21 may directly acquire at least one of the first data 61 and the second data 62 from the corresponding measurement device, or may acquire the data as a simulation execution result. In another example, the controller 21 may acquire at least one of the first data 61 and the second data 62 from a storage area of another computer or an external storage device via, for example, a network, the storage medium 92, or the like. Here, in a case where both data are acquired, the first data 61 and the second data 62 may be stored in the same storage area (storage device and storage medium), or may be stored in different storage areas. The number of samples of at least one of the first data 61 and the second data 62 to be acquired may be selected as appropriate according to the embodiment.

Upon acquiring at least one of the first data 61 and the second data 62 for each target material, the controller 21 advances the process to the next step S202.

### (Step S202)

In step S202, the controller 21 operates as the conversion unit 212, and executes at least one of a process of converting the acquired first data 61 into the first feature vector 71 and a process of converting the acquired second data 62 into the second feature vector 72 by using at least one of the trained first encoder 51 and the trained second encoder 52.

Specifically, in a case where the first data 61 is acquired and the acquired first data 61 is converted into the first feature vector 71, the controller 21 refers to the learning result data 125 to set the trained first encoder 51. Then, the controller 21 inputs the first data 61 for each target material to the trained first encoder 51 and executes arithmetic processing for the trained first encoder 51. As a result of this arithmetic processing, the controller 21 acquires the first feature vector 71 of each target material.

Similarly, in a case where the second data 62 is acquired and the acquired second data 62 is converted into the second feature vector 72, the controller 21 refers to the learning result data 125 to set the trained second encoder 52. Then, the controller 21 inputs the second data 62 for each target material to the trained second encoder 52 and executes arithmetic processing for the trained second encoder 52. As a result of this arithmetic processing, the controller 21 acquires the second feature vector 72 of each target material.

Upon acquiring at least one of the first feature vector 71 and the second feature vector 72 of each target material by the above process, the controller 21 advances the process to the next step S203.

### (Step S203)

In step S203, the controller 21 operates as the output processing unit 215, and maps each value of at least one of the obtained first feature vector 71 and second feature vector 72 of each target material onto the space VS. The space VS is for displaying the positional relationship of the feature vector.

In one example, the controller 21 may directly map each value of at least one of the obtained first feature vector 71 and second feature vector 72 of each target material onto the space VS. In another example, the controller 21 may convert each value of at least one of the obtained first feature vector 71 and second feature vector 72 of each target material into a low dimension to maintain a positional relationship of each of the values, and then map each converted value onto the space VS. As an example of the conversion, the original dimension of each feature vector (71, 72) may be about several tens to a thousand. In contrast, the converted dimension may be two-dimensional or three-dimensional. The conversion method is not particularly limited as long as the positional relationship of the feature vector can be maintained as much as possible, and may be selected as appropriate according to the embodiment. As the conversion method, for example, t-distributed stochastic neighbor embedding (t-SNE), non-negative matrix factorization (NMF), principal component analysis (PCA), independent component analysis (ICA), fast algorithm for ICA (a fast ICA), multidimensional scaling (MDS), spectral embedding, random projection, uniform manifold approximation and projection (UMAP), or the like may be adopted. The space VS, where each converted value is mapped, may be referred to as, for example, a visualization space, a low-dimensionalized feature space, or the like.

When the mapping of each feature vector onto the space VS is completed, the controller 21 advances the process to the next step S204.

### (Step S204)

In step S204, the controller 21 operates as the output processing unit 215, and outputs each value of at least one of the first feature vector 71 and the second feature vector 72 of each target material, each value having been mapped onto the space VS. When each value of at least one of the first feature vector 71 and the second feature vector 72 is converted to a low dimension in the process of step S203, the controller 21 outputs each value of the feature vector converted to a low dimension.

Each of the output destination and the output format may be selected as appropriate according to the embodiment. The output destination may be, for example, the output device 26, an output device of another computer, or the like. The output format may be, for example, screen output, printing, or the like. The controller 21 may execute any information processing when outputting the feature vector. As an example of the information processing, the controller 21 may receive the selection of one or more materials of interest from among a plurality of target materials. The material of interest may be selected, for example, by a method of specifying from a list of target materials, a method of specifying a feature vector that is displayed on the space VS, or other methods. Then, the controller 21 may output the selected material of interest separately from other target materials. Further, the controller 21 may output a list of other target materials for feature vectors present in the proximity range of the feature vector of the selected material of interest. The proximity range may be specified as appropriate. The other target materials present in the proximity range may be output after being sorted in the order of proximity on the space VS.

When the output of each value of the feature vector is completed, the controller 21 ends the process procedure related to data presentation according to the present operation example. Note that the controller 21 may repeatedly execute the processes of the above steps S201 to S204 at any timing, such as when receiving a command from an operator. At the time of this repetition, at least a part of the data (at least one of the first data 61 and the second data 62), acquired in step S201, may be subjected to change, correction, addition, deletion, or the like, as appropriate. As a result, the data output in step S204 may be changed.

(B) Process of Generating Second Data from First Data

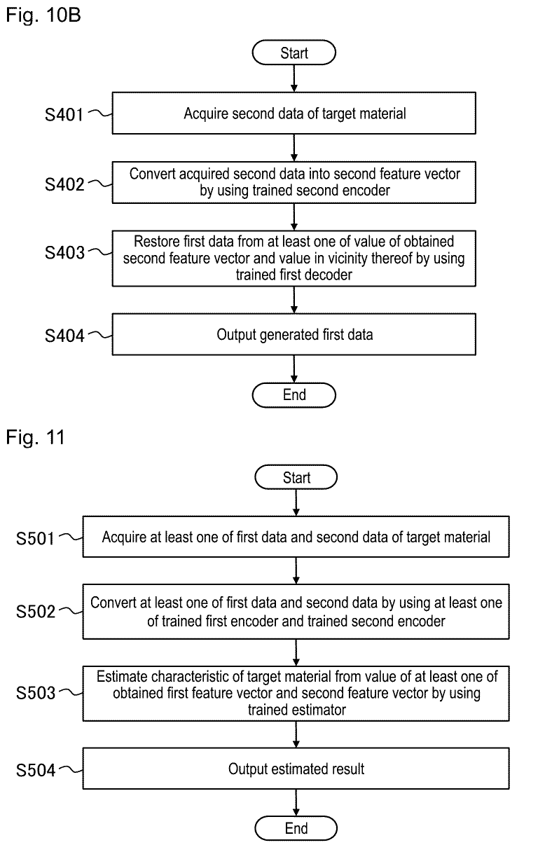

FIG. 10A is a flowchart illustrating an example of a process procedure for generating the second data 64 from the first data 63 by the data processing device 2 according to the present embodiment. The following process procedure related to data generation is an example of a data generation method. The command portion of the following data generation process procedure in the data processing program 82 is an example of the data generation program. However, the following process procedure related to data generation is merely an example, and each step may be changed as much as possible. In addition, with respect to the following process procedure related to data generation, steps can be omitted, replaced, and added as appropriate according to the embodiment.

### (Step S301)

In step S301, the controller 21 operates as the target data acquisition unit 211, and acquires the first data 63 for at least one or more target materials. The first data 63 is of the same type as the first data 31 for learning. Similarly to the first data 31, the first data 63 may be obtained by actual measurement or may be obtained by simulation. The number of first data 63 to be acquired may be determined as appropriate according to the embodiment.

In one example, the controller 21 may directly acquire the first data 63 from the measurement device, or may acquire the first data as a simulation execution result. In another example, the controller 21 may acquire the first data 63 from a storage area of another computer or an external storage device via, for example, a network, the storage medium 92, or the like. Upon acquiring the first data 63, the controller 21 advances the process to the next step S302.

### (Step S302)

In step S302, the controller 21 operates as the conversion unit 212, and converts the acquired first data 63 into the first feature vector 73 by using the trained first encoder 51. Specifically, the controller 21 refers to the learning result data 125 to set the trained first encoder 51. The controller 21 inputs the acquired first data 63 to the trained first encoder 51 and executes arithmetic processing for the trained first encoder 51. As a result of this arithmetic processing, the controller 21 acquires the first feature vector 73 of the target material. Upon acquiring the first feature vector 73, the controller 21 advances the process to the next step S303.

### (Step S303)

In step S303, the controller 21 operates as the restoration unit 213, and restores the second data 64 from at least one of the value of the first feature vector 73 obtained by the conversion and the value in the proximity thereof by using the trained second decoder 56. That is, the controller 21 handles at least one of the value of the first feature vector 73 and the value in the proximity thereof obtained by the process of step S302 as the value of the second feature vector, thereby executing the restoration of the second data 64.

Specifically, the controller 21 refers to the learning result data 125 to set the trained second decoder 56. Further, the controller 21 determines one or more input values to the trained second decoder 56 from the value of the first feature vector 73 obtained by the process of step S302 and the proximity range thereof. The proximity range may be set as appropriate. As an example, the trained first encoder 51 and the trained second encoder 52 may be used to calculate the maximum value of the first distance of the position sample. The proximity range may be set based on the maximum value of the first distance. The controller 21 may use the value of the obtained first feature vector 73 as it is as an input value, or may use a proximity value of the obtained first feature vector 73 as an input value. The proximity value may be determined as appropriate from the proximity range of the first feature vector 73.

Then, the controller 21 inputs the determined input value to the trained second decoder 56 and executes arithmetic processing for the trained second decoder 56. As a result of this arithmetic processing, the controller 21 can generate the second data 64 for the target material (i.e., acquire the restored second data 64 from the trained second decoder 56). In the process of step S303, one or more pieces of second data 64 may be generated for one piece of first data 63 by selecting one or more input values. Upon generating the second data 64, the controller 21 advances the process to the next step S304.

### (Step S304)

In step S304, the controller 21 operates as the output processing unit 215, and outputs the generated second data 64. Each of the output destination and the output format may be selected as appropriate according to the embodiment. The output destination may be, for example, the RAM, the storage 22, the output device 26, an output device of another computer, a storage area of another computer, or the like. The output format may be, for example, data output, screen output, printing, or the like.

When the output of the generated second data 64 is completed, the controller 21 ends the process procedure related to data generation according to the present operation example. Note that the controller 21 may repeatedly execute the processes of the above steps S301 to S304 at any timing, such as when receiving a command from the operator. At the time of this repetition, in the process of step S301, the first data 63 to be processed may be selected as appropriate.

(C) Process of Generating First Data from Second Data

FIG. 10B is a flowchart illustrating an example of a process procedure for generating the first data 66 from the second data 65 by the data processing device 2 according to the present embodiment. The following process procedure related to data generation is an example of a data generation method. The command portion of the following data generation process procedure in the data processing program 82 is an example of the data generation program. However, the following process procedure related to data generation is merely an example, and each step may be changed as much as possible. In addition, with respect to the following process procedure related to data generation, steps can be omitted, replaced, and added as appropriate according to the embodiment.

### (Step S401)

In step S401, the controller 21 operates as the target data acquisition unit 211, and acquires the second data 65 for at least one or more target materials. The second data 65 is of the same type as the second data 32 for learning. Similarly to the second data 32, the second data 65 may be obtained by actual measurement or may be obtained by simulation. The number of second data 65 to be acquired may be determined as appropriate according to the embodiment.

In one example, the controller 21 may directly acquire the second data 65 from the measurement device, or may acquire the second data as a simulation execution result. In another example, the controller 21 may acquire the second data 65 from a storage area of another computer or an external storage device via, for example, a network, the storage medium 92, or the like. Upon acquiring the second data 65, the controller 21 advances the process to step S402.

### (Step S402)

In step S402, the controller 21 operates as the conversion unit 212, and converts the acquired second data 65 into the second feature vector 75 by using the trained second encoder 52. Specifically, the controller 21 refers to the learning result data 125 to set the trained second encoder 52. The controller 21 inputs the acquired second data 65 to the trained second encoder 52 and executes arithmetic processing for the trained second encoder 52. As a result of this arithmetic processing, the controller 21 acquires the second feature vector 75 of the target material. Upon acquiring the second feature vector 75, the controller 21 advances the process to the next step S403.

### (Step S403)

In step S403, the controller 21 operates as the restoration unit 213, and restores the first data 66 from at least one of the value of the second feature vector 75 obtained by the conversion and the value in the proximity thereof by using the trained first decoder 55. That is, the controller 21 handles at least one of the value of the second feature vector 75 obtained by the process of step S402 and the value in the proximity thereof as the value of the first feature vector, thereby executing the restoration of the first data 66.

Specifically, the controller 21 refers to the learning result data 125 to set the trained first decoder 55. Further, the controller 21 determines one or more input values to the trained first decoder 55 from the value of the second feature vector 75 obtained by the process of step S402 and the proximity range thereof. Similarly to the above step S303, the proximity range may be set as appropriate. As an example, the proximity range may be set based on the maximum value of the first distance calculated by the trained first encoder 51 and the trained second encoder 52. The controller 21 may use the value of the obtained second feature vector 75 as it is as an input value, or may use a proximity value of the obtained second feature vector 75 as an input value. The proximity value may be determined as appropriate from the proximity range of the second feature vector 75.

Then, the controller 21 inputs the determined input value to the trained first decoder 55 and executes arithmetic processing for the trained first decoder 55. As a result of this arithmetic processing, the controller 21 can generate the first data 66 for the target material (i.e., acquire the restored first data 66 from the trained first decoder 55). In the process of step S403, one or more pieces of first data 66 may be generated for one piece of second data 65 by selecting one or more input values. Upon generating the first data 66, the controller 21 advances the process to the next step S404.

### (Step S404)

In step S404, the controller 21 operates as the output processing unit 215, and outputs the generated first data 66. Each of the output destination and the output format may be selected as appropriate according to the embodiment. The output destination may be, for example, the RAM, the storage 22, the output device 26, an output device of another computer, a storage area of another computer, or the like. The output format may be, for example, data output, screen output, printing, or the like.

When the output of the generated first data 66 is completed, the controller 21 ends the process procedure related to data generation according to the present operation example. Note that the controller 21 may repeatedly execute the processes of the above steps S401 to S404 at any timing, such as when receiving a command from the operator. At the time of this repetition, in the process of step S401, the second data 65 to be processed may be selected as appropriate.

### (D) Characteristic Estimation Process

FIG. 11 is a flowchart illustrating an example of a process procedure related to characteristic estimation for a target material by the data processing device 2 according to the present embodiment. The following process procedure related to characteristic estimation is an example of an estimation method. The command portion of a process procedure related to the following characteristic estimation in the data processing program 82 is an example of the estimation program. However, the following process procedure related to characteristic estimation is merely an example, and each step may be changed as much as possible. In addition, with respect to the following process procedure related to characteristic estimation, steps can be omitted, replaced, and added as appropriate according to the embodiment.

### (Step S501)

In step S501, the controller 21 operates as the target data acquisition unit 211, and acquires at least one of first data 67 and second data 68 regarding the crystal structure of the target material. The first data 67 and the second data 68 are of the same type as the first data 31 and the second data 32 for learning. Similarly to the first data 31 and the second data 32, the first data 67 and the second data 68 may be obtained by actual measurement or may be obtained by simulation.

In one example, the controller 21 may directly acquire at least one of the first data 67 and the second data 68 from the corresponding measurement device, or may acquire the data as a simulation execution result. In another example, the controller 21 may acquire at least one of the first data 67 and the second data 68 from a storage area of another computer or an external storage device via, for example, a network, the storage medium 92, or the like. Upon acquiring at least one of the first data 67 and the second data 68 for the target material, the controller 21 advances the process to the next step S502.

### (Step S502)

In step S502, the controller 21 operates as the conversion unit 212, and executes at least one of a process of converting the first data 67 acquired using the trained first encoder 51 into the first feature vector 77 and a process of converting the second data 68 acquired using the trained second encoder 52 into the second feature vector 78.

Specifically, when the trained estimator 58 is configured to estimate the characteristic of the target material from the first feature vector, the controller 21 refers to the learning result data 125 to set the trained first encoder 51. The controller 21 inputs the acquired first data 67 to the trained first encoder 51 and executes arithmetic processing for the trained first encoder 51. As a result of this arithmetic processing, the controller 21 acquires the first feature vector 77 of the target material.

Similarly, when the trained estimator 58 is configured to estimate the characteristic of the target material from the second feature vector, the controller 21 refers to the learning result data 125 to set the trained second encoder 52. The controller 21 inputs the acquired second data 68 to the trained second encoder 52 and executes arithmetic processing for the trained second encoder 52. As a result of this arithmetic processing, the controller 21 acquires the second feature vector 78 of the target material.

Upon acquiring at least one of the first feature vector 77 and the second feature vector 78 of the target material by the above process, the controller 21 advances the process to the next step S503.

### (Step S503)

In step S503, the controller 21 operates as the estimation unit 214, and estimates the characteristic of the target material from the value of at least one of the obtained first feature vector 77 and second feature vector 78 by using the trained estimator 58. Specifically, the controller 21 refers to the learning result data 125 to set the trained estimator 58. The controller 21 inputs the value of at least one of the acquired first feature vector 77 and second feature vector 78 to the trained estimator 58 and executes arithmetic processing for the trained estimator 58. As a result of this arithmetic processing, the controller 21 acquires an output value corresponding to the result of estimating the characteristic of the target material from the trained estimator 58. Upon acquiring the estimation result, the controller 21 advances the process to step S504.

### (Step S504)

In step S504, the controller 21 operates as the output processing unit 215, and outputs information regarding a result of estimating the characteristic of the target material. Each of the output destination and the output format may be selected as appropriate according to the embodiment. The output destination may be, for example, the RAM, the storage 22, the output device 26, an output device of another computer, a storage area of another computer, or the like. The output format may be, for example, data output, screen output, voice output, printing, or the like.

When the output of the result of estimating the characteristic of the target material is completed, the controller 21 ends the process procedure related to the characteristic estimation according to the present operation example. Note that the controller 21 may repeatedly execute the processes of the above steps S501 to S504 at any timing, such as when receiving a command from the operator. At the time of this repetition, in the process of step S501, at least one of the first data 67 and the second data 68 to be processed may be selected as appropriate.

### [Features]

As described above, in the present embodiment, a positive sample and a negative sample to be used for machine learning can be prepared depending on whether or not the materials are the same. Therefore, the model generation device 1 can generate the trained first encoder 51 and the trained second encoder 52 at low cost by the above steps S101 and S102. In the data processing device 2, by the processes of the above steps S201 to S204, at least one of the first data 61 and the second data 62 for each of the plurality of target materials can be mapped onto a feature space by using at least one of the trained first encoder 51 and the trained second encoder 52. In this feature space, the similarity of materials can be evaluated by the positional relationship of the feature vector. Based on this evaluation result, new knowledge of the material can be obtained.

In the present embodiment, data indicating the property of the material may be adopted as the first data 31 based on the local perspective of the crystal structure, and data indicating the property of the material may be adopted as the second data 32 based on the overall overhead perspective. As a result, it is possible to generate trained encoders (51, 52) that have acquired the ability to map the respective data onto the feature space where the similarity of materials can be evaluated from both the local and overhead perspectives. In the data processing device 2, new knowledge of the material can be obtained more accurately by using at least one of the trained first encoder 51 and the trained second encoder 52 in the processes of the above steps S201 to S204.

In the present embodiment, the model generation device 1 can generate the trained first decoder 55 that has acquired the ability to restore the first data by the process of the above step S103. As a result, the data processing device 2 can generate valid first data from the second data for the target material with respect to the material known in the second data but unknown in the first data by using the generated trained second encoder 52 and trained first decoder 55 by the processes of the above steps S401 to S403.

In the present embodiment, the model generation device 1 can generate the trained second decoder 56 that has acquired the ability to restore the second data by the process of the above step S103. As a result, the data processing device 2 can generate valid second data from the first data for the target material with respect to the material known in the first data but unknown in the second data by using the generated trained first encoder 51 and trained second decoder 56 by the processes of the above steps S301 to S303.

In the present embodiment, the model generation device 1 can generate the trained estimator 58 that has acquired the ability to estimate the characteristic of the material from at least one of the first feature vector and the second feature vector by the process of the above step S104. As a result, the data processing device 2 can estimate the characteristic of the target material from at least one of the first data and the second data by using at least one of the trained first encoder 51 and the trained second encoder 52 and the trained estimator 58 by the processes of the above steps S501 to S503.

In the present embodiment, the correct information 35 may be provided for all materials for learning, but information regarding the similarity of materials is included in the feature space where mapping is performed by each trained encoder (51, 52). The estimator 58 is configured to estimate the characteristic of the material from the feature vector on the feature space, and can thus consider the above information when estimating the characteristic of the material. It is thus possible to generate the trained estimator 58 capable of accurately estimating the characteristics of the materials without providing the correct information 35 for all materials. Therefore, according to the present embodiment, the trained estimator 58 capable of accurately estimating the characteristic of the material can be generated at low cost.

### § 4 Modifications

Although the embodiment of the present invention has been described in detail above, the above description is merely an example of the present invention in all respects. It goes without saying that various improvements or modifications can be made without departing from the scope of the present invention. For example, the following changes can be made. Hereinafter, the same reference numerals are used for the same components as those in the above embodiment, and the same point as in the above embodiment is omitted from the description as appropriate. The following modifications can be combined as appropriate.

<4.1>

In the above embodiment, the terms “first” and “second” are referred to with respect to the data, encoder, and decoder. However, these references do not indicate that the number of these components is limited to two. That is, “third” and subsequent data, encoders, and decoders may appear.

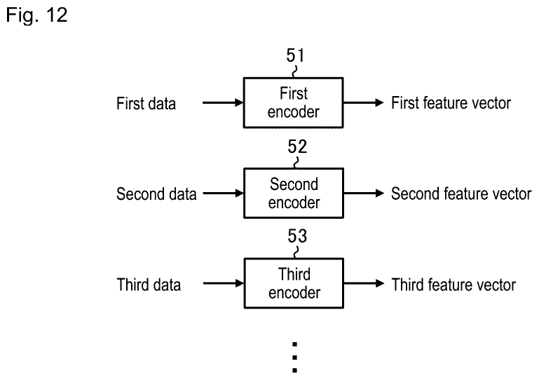

FIG. 12 schematically illustrates an example of a configuration of an encoder according to another mode as an example of a scene in which “third” and subsequent components appear. In the example of FIG. 12, in addition to the first encoder 51 and the second encoder 52, there is a third encoder 53 configured to convert third data into a third feature vector having the same dimension as the first feature vector and the second feature vector. Similarly to the first data and the second data, the third data indicates information regarding the crystal structure of the material.

In the present modification, the model generation device 1 may acquire a plurality of types of data regarding the crystal structure of the material. Each type of data may indicate a property of the material with an index different from other types of data. The plurality of types of acquired learning data may include a plurality of positive samples and negative samples. Each positive sample may include a combination of a plurality of types of data for the same material. Each negative sample may include at least one of a plurality of types of data for a material different from the material of the corresponding position sample.

The model generation device 1 may perform machine learning for a plurality of encoders by using a plurality of types of acquired data. At least one encoder may correspond to each type of data. Each encoder may be configured to correspond to any type of a plurality of types of data, and convert the corresponding type of data into a feature vector having the same dimension as the other encoders. The machine learning for the plurality of encoders may be configured by training the plurality of encoders so that the values of the plurality of feature vectors calculated from the plurality of types of data of each positive sample are positioned close to each other by using the respective encoders, and the value of the feature vector calculated from at least one of the plurality of types of data of each negative sample is positioned far from the value of at least one of the plurality of feature vectors calculated from the corresponding positive sample. Each of the first data 31 and the second data 32 in the above embodiment may be any of the plurality of types of data. Each of the first encoder 51 and the second encoder 52 may be any of the plurality of encoders.

The data processing device 2 may acquire at least one of the plurality of types of data regarding the crystal structure of each of the plurality of target materials. The data processing device 2 may convert at least one of the plurality of types of acquired data for each target material into a feature vector by using at least one of the plurality of trained encoders. The data processing device 2 may map the value of the obtained feature vector of each target material onto the space VS and output each value of the feature vector mapped onto the space VS.

The model generation device 1 may perform machine learning for at least one decoder corresponding to each encoder. The machine learning for at least one decoder may be configured by training at least one decoder so that a result of at least one decoder restoring the data for the corresponding type from the feature vector, calculated from the data for the corresponding type by using the corresponding encoder, matches the data for the corresponding type. Correspondingly, the data processing device 2 may generate other data (above first data 63/second data 65) from the target data (second data 64/first data 66) among the plurality of types of data.

The model generation device 1 may generate a trained estimator that has acquired an ability to estimate a characteristic of a material from at least one of a plurality of feature vectors by machine learning. The machine learning for the estimator may be configured by training the estimator so that a result of the estimator estimating a characteristic of a material from at least one of a plurality of feature vectors, calculated from at least one of a plurality of types of data by using at least one of a plurality of encoders, matches a true value indicated by corresponding correct information. Correspondingly, the data processing device 2 may estimate the characteristic of the target material from at least one of the plurality of types of data.

<4.2>

In the data processing device 2 according to the above embodiment, at least one of the data presentation process, the process of generating the second data from the first data, the process of generating the first data from the second data, and the estimation process may be omitted.

In a case where the process of generating the second data from the first data is omitted, the process of generating the trained second decoder 56 in step S103 may be omitted in the model generation device 1. Information regarding the trained second decoder 56 may be omitted from the learning result data 125.

In a case where the process of generating the first data from the second data is omitted, the process of generating the trained first decoder 55 in step S103 may be omitted in the model generation device 1. Information regarding the trained first decoder 55 may be omitted from the learning result data 125.

In a case where the estimation process is omitted, the process of generating the trained estimator 58 in the model generation device 1 (step S104) may be omitted. Information regarding the trained estimator 58 may be omitted from the learning result data 125.

In a case where the trained first encoder 51 is not used in the data processing device 2, information regarding the trained first encoder 51 may be omitted from the learning result data 125. In a case where the trained second encoder 52 is not used in the data processing device 2, the information regarding the trained second encoder 52 may be omitted from the learning result data 125.

In response to the omission of each process, components for executing the corresponding process may be omitted in each software module of the model generation device 1 and the data processing device 2. As an example, in a case where the data presentation process is omitted, a portion related to the data presentation process for the target data acquisition unit 211, the conversion unit 212, and the output processing unit 215 may be omitted in the software configuration of the data processing device 2. As another example, in a case where both the data generation processes are omitted, the portion for generating the trained first decoder 55 and the trained second decoder 56 may be omitted in the software configuration of the model generation device 1. In the software configuration of the data processing device 2, the portion related to the data generation process for the target data acquisition unit 211, the conversion unit 212, and the output processing unit 215 and the restoration unit 213 may be omitted. As another example, in a case where the estimation process is omitted, the portion for generating the trained estimator 58 may be omitted in the software configuration of the model generation device 1. In the software configuration of the data processing device 2, the portion related to the estimation process for the target data acquisition unit 211, the conversion unit 212, and the output processing unit 215 and the estimation unit 214 may be omitted.

At least one of the data presentation process, the process of generating the second data from the first data, the process of generating the first data from the second data, and the estimation process may be executed by another computer. As an example, the data presentation process, the process of generating the second data from the first data, the process of generating the first data from the second data, and the estimation process may be executed by different computers. In this case, the computer that executes each process may be configured in the same manner as the data processing device 2.

<4.3>

In the above embodiments, the trained estimator 58 is generated. Corresponding to this trained estimator 58, a trained converter may be generated to estimate at least one of the first feature vector and the second feature vector from the information indicative of the characteristic of the target material. The trained converter can be generated by machine learning with the input and output of the estimator 58 reversed. That is, the machine learning for the converter may be configured by training the converter so that at least one of the first feature vector and the second feature vector, estimated by the converter from the characteristic indicated by the correct information 35, matches at least one of the first feature vector and the second feature vector, calculated from at least one of the corresponding first data 31 and second data 32 by using at least one of the first encoder 51 and the second encoder 52. The trained converter may be generated by the model generation device 1 or may be generated by another computer.

Thereby, at least one of the first feature vector and the second feature vector of the material with the characteristic of the target may be estimated from the information indicating the characteristic of the target by using the trained converter. Then, at least one of the first data and the second data may be restored from at least one of the estimated first feature vector and second feature vector by using at least one of the trained first decoder 55 and the trained second decoder 56. The process of restoring data from the characteristic of the material may be executed by the data processing device 2 or may be executed by another computer.

### § 5 Experimental Examples

To verify the effectiveness of the present invention, a trained first encoder and a trained second encoder according to each of the following experimental examples were generated. However, the present invention is not limited to the following experimental examples.

### (1) First Experimental Example

First, 122,543 pieces of inorganic material data containing five or fewer types of elements were collected (downloaded) from inorganic material data registered in the Materials Project database (https://materialsproject.org/). Three-dimensional atomic position data included in the collected inorganic material data was adopted as first data. X-ray diffraction data obtained from the three-dimensional atomic position data by simulation according to Bragg's law (using Python library “pymatgen”) was adopted as second data. Then, a trained first encoder and a trained second encoder according to a first experimental example were generated by a method similar to that of the above embodiment. As the first encoder, a convolutional neural network with a convolution layer was adopted (reference literature: Charles R. Qi, Li Yi Hao Su, Leonidas J. Guibas, “PointNet++: Deep Hierarchical Feature Learning on Point Sets in a Metric Space”, 31st Conference on Neural Information Processing Systems (NIPS 2017)/Tian Xie, Jeffrey C. Grossman, “Crystal Graph Convolutional Neural Networks for an Accurate and Interpretable Prediction of Material Properties”, Phys. Rev. Lett. 120, 145301, 6 Apr. 2018). A one-dimensional convolutional neural network was adopted as the second encoder. Both encoders were configured to convert the respective data into a 1024-dimensional feature vector. Triplet Loss was adopted as a loss function in machine learning for each encoder. Specifically, an error L was calculated using Equations 1 to 3 below, and the parameters of each encoder were optimized by backpropagation.

$$\begin{matrix}[ Equation 1 ] & \\ {L}_{nx}^{( i )} ( {x}_{i} , {y}_{i} , {x}_{i}^{′} ) = max ( 0 , ‖ {x}_{i} - {y}_{i} ‖ - ‖ {x}_{i}^{′} - {y}_{i} ‖ + m ) & ( Equation 1 )\end{matrix}$$

$$\begin{matrix}[ Equation 2 ] & \\ {L}_{ny}^{( i )} ( {x}_{i} , {y}_{i} , {y}_{i}^{′} ) = max ( 0 , ‖ {x}_{i} - {y}_{i} ‖ - ‖ {x}_{i} - {y}_{i}^{′} ‖ + m ) & ( Equation 2 )\end{matrix}$$

$$\begin{matrix}[ Equation 3 ] & \\ L = \frac{1}{2 N} {\sum }_{i = 1}^{N} ( {L}_{nx}^{( i )} - {L}_{ny}^{( i )} & ( Equation 3 )\end{matrix}$$

Note that x represents the first data, and y represents the second data. The combination of (xᵢ, yᵢ) indicates a positive sample. xi′ and yi′ each represent a negative sample.

By using the generated trained first encoder, the first data (three-dimensional atomic position data) for each material used for machine learning was converted into a first feature vector. Next, t-SNE was used to convert the dimension of each first feature vector from 1024 dimensions to two dimensions, map the value of each feature vector onto a two-dimensional visualization space, and perform screen output. Then, the obtained map (data distribution) was analyzed by two methods: (A) analysis of global distribution and (B) analysis of local proximity.

### (A) Analysis of Global Distribution

To confirm how elements corresponding to the respective materials are distributed on the map, analysis was performed for the range in which elements corresponding to materials containing the respective elements of the periodic table are present in the obtained map. In addition, in the obtained map, each element according to the value of the physical characteristic is color-coded to perform analysis for the correspondence between the distribution of each element and the physical characteristics (energy above the hull, band gap, magnetization).

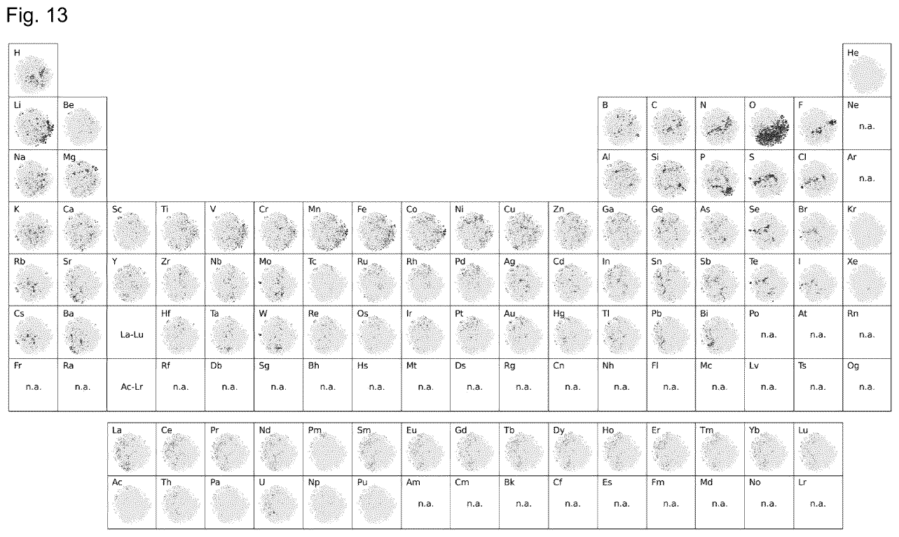

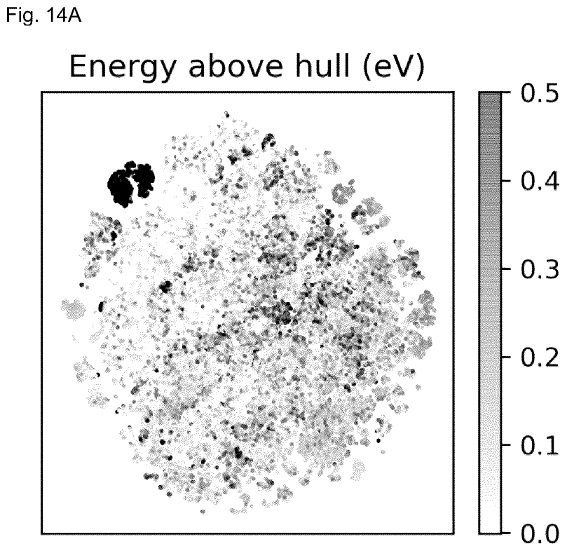

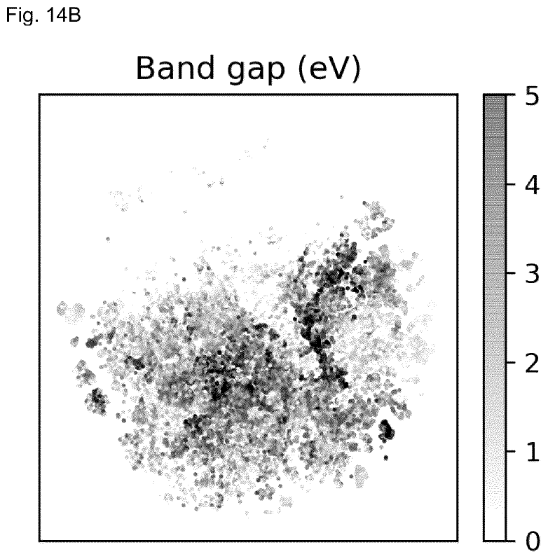

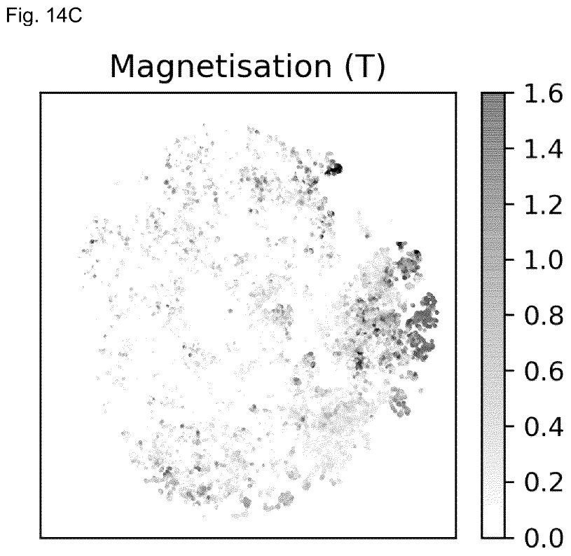

FIG. 13 illustrates a result of checking a range in which elements corresponding to materials containing the respective elements of the periodic table are present in the obtained map. FIGS. 14A to 14C illustrate results of color-coding each element according to the values of the physical characteristics (FIG. 14A: energy above the hull, FIG. 14B: band gap, FIG. 14C: magnetization) in the obtained map. Note that “n.a.” in FIG. 13 indicates that there is no corresponding element.

As illustrated in FIG. 13, the presence ranges of the elements corresponding to the materials containing the respective elements were similar in each of the vertical direction and the horizontal direction of the periodic table. This result showed that the obtained map appropriately grasps the similarity of the behavior of the elements in the respective materials. In addition, as illustrated in FIGS. 14A to 14C, elements with similar physical characteristic formed a cluster on the obtained map. For example, as illustrated in FIG. 14A, a cluster of unstable compounds with a large energy value was identified in the upper left portion of the map. In addition, the results of FIGS. 14B and 14C, it was confirmed that substances with similar band gaps or magnetization values form a plurality of clusters, and each cluster is a group of substances with similar structures or compositions. For example, in the result of FIG. 14B, it was confirmed that metals with low band gaps and non-metals with high band gaps form large clusters, respectively, throughout the map. Further, in the result of FIG. 14C, a cluster of rare earth permanent magnet materials with strong magnetizing properties was identified in the upper right portion of the map. These results show that the obtained map appropriately grasps the similarity of the physical characteristic of each material.

### (B) Analysis of Local Proximity

Next, to confirm what elements are arranged in the proximity of each element on the obtained map (i.e., whether the map captures the similarity of materials), each of two selected materials “Hg-1223 (HgBa₂Ca₂Cu₃O₈)” and “LiCoO₂” was used as a query, and materials present in the proximity of the query were searched.

In addition, two types of descriptors “Ewald Sum Matrix” and “Sine Coulomb Matrix”, proposed in the reference literature “Faber, F., Lindmaa, A., von Lilienfeld, O. A. & Armiento, R. ‘Crystal structure representations for machine learning models of formation energies’, Int. J. Quantum Chem. 115, 1094-1101 (2015)”, were used to generate feature vectors (feature amount representation of each material) according to a first comparative example and a second comparative example. This feature vector was generated by calculating an eigenvalue vector in which eigenvalues of a matrix are arranged in descending order of absolute values from two types of descriptors each represented by a matrix. Then, the material present in the proximity of each query was searched using the feature vector according to each comparative example.

TABLE 1

| | Our embedding | | Ewald Scrum Matrix | | Sine Coulomb Matrix | |
|---|---|---|---|---|---|---|
| No. | Formula | ID | Formula | ID | Formula | ID |
| Query | Ba2Ca2Cu3HgO8 | mp-22601 | Ba2Ca2Cu3HgO8 | mp-22601 | Ba2Ca2Cu3HgO8 | mp-22601 |
| 1 | Ba2Ca0Cu4HgO18 | mp-1228679 | [symbol] | mp-1218464 | T [symbol] | mp-909204 |
| 2 | Ba2CaCu2Hg8 | mp-6879 | Ba2 [symbol] O11 | mp-1214655 | CaLa2 [symbol] O6 | mvc-15176 |
| 3 | Ba4CaCu [symbol] | mvc-15237 | OsY4Mg5 | mp-1228574 | PtC4 [symbol] O12 | mp-1102940 |
| 4 | Va2Ca0Ti2 [symbol] | mp-66857 | Ba [symbol] | mp-558119 | Ba [symbol] O6 | mp-31758 |
| 5 | Ba4Ca4 [symbol] O17 | mp-1223285 | Ba [symbol] | mp-554949 | Hg [symbol] | mp-764065 |
| 6 | Ba2CaTi2 [symbol] | mp-6885 | Ba2N [symbol] Ti2Cu2O11 | mp-567043 | Ba2CuW08 | mp-905818 |
| 7 | Ba2Ca3 [symbol] O11 | mp-1228589 | Ba49 [symbol] O15 | mp-1223157 | CaLa2WO6 | mvc-15479 |
| 8 | Ba2Ca3M [symbol] Ti2O12 | mvc-139 | La [symbol] | mp-18573 | Ba2YT [symbol] O6 | mp-12349 |
| 9 | Ba2Mg3M [symbol] Ti2O18 | mvc-63 | Z [symbol] | mp1215364 | TiC [symbol] 2 | mp-998919 |
| 10 | Ba2YCu3P [symbol] | mp-1228257 | N [symbol] | mp-1103877 | Ti [symbol] 2 | mp-1005374 |
| 11 | Ba2C [symbol] | mvc-122 | Y4T [symbol] O21 | mp-129208 | LaT [symbol] 2 | mp-867817 |
| 12 | Ba2C [symbol] | mp-558733 | [symbol] | mp-1219109 | [symbol] | mp-973498 |
| 13 | BaCa3 [symbol] 4 | mvc-145 | Ba3 [symbol] | mp-1209141 | C [symbol] 2 | mp-867298 |
| 14 | Ba2Mg3Ti2 [symbol] 4 | mvc-29 | Ag [symbol] | mp-997108 | C [symbol] | mp-541753 |
| 15 | Ba2YCu2HgO7 | mp-12114583 | Y [symbol] 4 | mp-1215523 | T [symbol] | mp-1187742 |
| 16 | Ba2Ca3Ti2 [symbol] | mvc-128 | Ca4C [symbol] | mp-1227562 | [symbol] | mp-1184857 |
| 17 | Ba2 [symbol] | mvc-128 | [symbol] 4 | mp-1223819 | Ca4C [symbol] | mp-1227562 |
| 18 | BasM [symbol] 4Ti2Zn3O12 | mvc-148 | [symbol] O12 | mp-1219457 | Cd3Pt | mp-1183641 |
| 19 | Ba2CaTiCu2O7 | mp-632902 | C [symbol] 7 | mp-1212875 | Ag3Au | mp-1183214 |
| 20 | [symbol] O7 | mp-1262318 | BawY [symbol] O7 | mvc-2994 | m [symbol] 3 | mp-1221739 |
| 21 | BawY [symbol] O7 | mvc-16175 | T [symbol] | mp-1217358 | NdT [symbol] Ag2 | mp-974782 |
| 22 | Ba2Ca2Tis [symbol] O16 | mvc-3027 | N [symbol] | mp-1209832 | [symbol] | mp-973801 |
| 23 | Ba2CaTi2 [symbol] 4 | mvc-11852 | Ba4 [symbol] | mp-1194514 | TiC [symbol] | mp-1093975 |
| 24 | BaCaTi2 [symbol] 4 | mvc-13047 | Ba [symbol] O17 | mp-556337 | C [symbol] | mp-1226474 |
| 25 | Ba2M [symbol] 4 | mvc-132 | Ba2 [symbol] O11 | mp-505228 | P [symbol] 2 | mp-862913 |
| 26 | Ba2Mga [symbol] 4 | mvc-28 | [symbol] | mp-1203018 | [symbol] | mp-12 [symbol] 3 |
| 27 | [symbol] 4 | mvc-189 | [symbol] 2 | mp-1225812 | N [symbol] | mp-1180317 |
| 28 | Ba2C [symbol] O12 | mvc-149 | Ag [symbol] | mp-1229041 | [symbol] 2 | mp- [symbol] 67 |
| 29 | Ba2Mg3 [symbol] 4 | mvc-10585 | [symbol] | mp-1105102 | [symbol] | mp-1183645 |
| 30 | Ba2 [symbol] 2 | mp-1214767 | N [symbol] | mp-1220441 | H [symbol] 3 | mp-1164658 |
| 31 | Ba2C [symbol] O10 | mvc [symbol] | [symbol] | mp-121155 | [symbol] Au | mp-1095757 |
| 32 | Ba2Ca [symbol] | mp-57 [symbol] | C [symbol] | mp-1103614 | [symbol] Ag2 | mp-862955 |
| 33 | Ba2CaCu2 [symbol] | mp-1232840 | Ca [symbol] | mp-555816 | [symbol] | mp-1113498 |
| 34 | Ba2Y [symbol] O7 | mp-654374 | Ca [symbol] [symbol] | mp-1207157 | VAg [symbol] | mp-1216423 |
| 35 | Ba2Ti2 [symbol] O12 | mvc-141 | T [symbol] | mp-1101632 | [symbol] | mp-1097125 |
| 36 | [symbol] | mp-555865 | [symbol] Au | mp-1219474 | P [symbol] | mp-970884 |
| 37 | Ba2YCu [symbol] 2 | mp-1228504 | [symbol] 11 | mp-1213463 | [symbol] | mp-1216611 |
| 38 | [symbol] | mp-1218930 | [symbol] 2O | mp-1078328 | Cd2AgPt | mp-1096169 |
| 39 | [symbol] Ca3Ti2V4O12 | mvc-160 | [symbol] N4 | mp-1224363 | [symbol] | mp-1113397 |
| 40 | Ba2A [symbol] O7 | mvc-13310 | MoN | mp-107 [symbol] | [symbol] | mp-1223808 |
| 41 | Ba2YCu [symbol] 2 | mp-1203772 | YZn [symbol] | mp-13160 | [symbol] AgPt | mp-1183537 |
| 42 | [symbol] O7 | mp-20824 | P [symbol] | mp-57130-2 | Ag [symbol] Au | mp-109 [symbol] |
| 43 | BawY [symbol] O7 | mvc-2992 | [symbol] | mp-1207133 | Ag3Au [symbol] 2 | mp-34982 |
| 44 | [symbol] | mp-664200 | N [symbol] O12 | mp-6706 [symbol] | P [symbol] | mp-662958 |
| 45 | [symbol] 2 | mp-1208972 | [symbol] O7 | mvc-280 | P [symbol] | mp-662950 |
| 46 | Ba2T [symbol] O11 | mp-506223 | N [symbol] O12 | mp-689927 | [symbol] | mp-1208792 |
| 47 | Ba2 [symbol] O7 | mp-1214738 | [symbol] | mp-1203482 | [symbol] Ag2Au | mp-1093948 |
| 48 | Ba2 [symbol] Mn4O11 | mp-867516 | [symbol] | mp-1208725 | Ag2 [symbol] 4 | mp-1229127 |
| 49 | [symbol] 2 | mp-1203753 | Ba [symbol] | mp-1228546 | [symbol] Au | mp-29 [symbol] 5 |
| 50 | Ba [symbol] | mp-1223790 | [symbol] 2 | mp-29113 | P [symbol] | mp- [symbol] 76 |

[symbol]

indicates data missing or illegible when filed

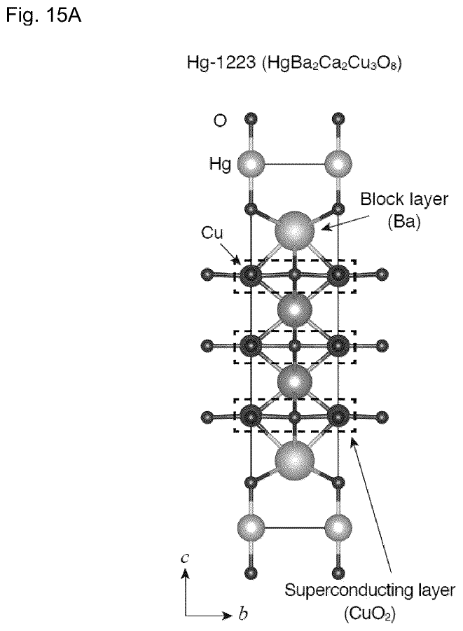

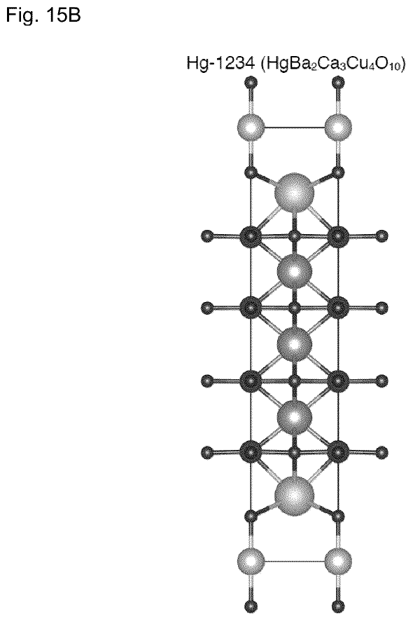

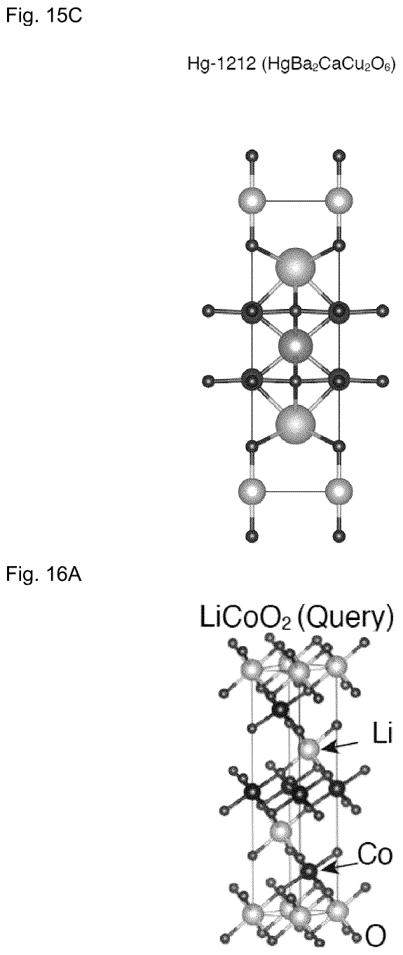

Table 1 shows materials extracted in the first to fiftieth proximities with respect to the query “Hg-1223” according to the first experimental example and each comparative example. FIG. 15A illustrates the composition of the query “Hg-1223”. FIG. 15B illustrates the composition of the material “Hg-1234 (HgBa₂Ca₃Cu₄O₁₀)” extracted in the closest (first) proximity according to the first experimental example. FIG. 15C illustrates the composition of the material “Hg-1212 (HgBa₂CaCu₂O₆)” extracted in the second proximity according to the first experimental example.

The query “Hg-1223” is the known superconductor with the highest critical temperature Tc. In the first experimental example, the superconductors “Hg-1234” and “Hg-1212” with high critical temperatures Tc were extracted in the first proximity and the second proximity of the query. As illustrated in FIGS. 15A to 15C, “Hg-1234” and “Hg-1212” extracted as the first proximity and the second proximity each have a structure similar to that of the query “Hg-1223”. In the first example, the TI-based superconductors “TI-2234” (fourth), “TI-2212” (sixth), “TI-1234” (seventh), and “TI-1212” (nineteenth) with high critical temperatures Tc were extracted. Further, in the first example, most of the materials extracted up to the fiftieth proximity were superconductors. In contrast, in the method of each comparative example, a relatively large number of materials, which were not superconductors but irrelevant, were extracted.

TABLE 2

| | Our embedding | | Ewald Scrum Matrix | | Sine Coulomb Matrix | |
|---|---|---|---|---|---|---|
| No. | Formula | ID | Formula | ID | Formula | ID |
| Query | LiCoO2 | mp-22526 | LiCoO2 | mp-22526 | LiCoO2 | mp-22526 |
| 1 | LiCuO2 | mp-25372 | LiNiO2 | mp-25597 | LiCoO2 | mp-1222334 |
| 2 | LiNiO2 | mp-25411 | Co(HO)2 | mp-24105 | CoHO2 | mp-27913 |
| 3 | LiRnO2 | mp-14115 | LiFeO2 | mp-1222302 | LiCoF2 | mp-1097040 |
| 4 | Li [symbol] aO2 | mp-8002 | LiNiO2 | mp-25316 | LiCoN | mp-1246462 |
| 5 | LiCuO2 | mp-754912 | Li2NiO2 | mp-19183 | Li2CoN2 | mp-1247124 |
| 6 | LiFeO2 | mp-19419 | MgMnN2 | mp-1247154 | Be5Co | mp-1071690 |
| 7 | LiZnO2 | mp-754344 | Li2CaC [symbol] | mp-1096283 | Be3Co | mp-1183423 |
| 8 | NiO2 | mp-25210 | NiO2 | mp-25210 | Be2Co | mp-1227342 |
| 9 | LiNiO2 | mp-25687 | LiFeO [symbol] | mp-775022 | CoCN | mp-1245659 |
| 10 | Li(NiO2)2 | mvc-16803 | MnO2 | mp-1221542 | Li3Co | mp-976017 |
| 11 | Li2Fe [symbol] O4 | mp-1222775 | Co(HO)2 | mp-625939 | Li2CoO2 | mp-755133 |
| 12 | LiCuO2 | mp-9158 | Co(HO)2 | mp-625943 | Li2CoO2 | mp-759207 |
| 13 | LiNiO2 | mp-1176588 | Li2CuO2 | mp-1239022 | Be12Co | mp-1104193 |
| 14 | LiNiO2 | mp-25316 | CoO2 | mp-1062939 | CoO2 | mp-1181781 |
| 15 | Li2CoO2 | mp-755133 | NaCoO2 | mp-1221066 | CoO2 | mvc-13108 |
| 16 | Li(CoO2)2 | mp-754742 | NiO2 | mp-634706 | Co(HO)2 | mp-628708 |
| 17 | Li2MnCoO4 | mp-1173885 | MgMnO2 | mp-1080243 | Co(HO)2 | mp-625938 |
| 18 | CoHO2 | mp-27913 | LiCuF2 | mp-753098 | Co(HO)2 | mp-625343 |
| 19 | Li(NiO2)2 | mp-755045 | Ni(HO)2 | mp-625074 | Co(HO)2 | mp-24105 |
| 20 | Li2CoNiO4 | mp-753276 | C [symbol] O2 | mp-1009555 | CoO2 | mp-1062339 |
| 21 | CoO2 | mp-82686 | CoHO2 | mp-27913 | CoO2 | mp-1062643 |
| 22 | LiNiO2 | mp-632864 | NaLi2A [symbol] | mp-1014873 | CoO2 | mp-55870 |
| 23 | Li(CoO2)3 | mp-762635 | LiNiO2 | mp-26411 | CoH3 | mp-1183678 |
| 24 | Li2FeO2 | mp-755094 | Li2CoO2 | mp-755133 | CoH | mp-1206874 |
| 25 | NaNiO2 | mp-578611 | LiCuO2 | mp-754912 | CoO2 | mp-1063268 |
| 26 | CoO2 | mp-162643 | C [symbol] N2 | mp-1014264 | CoN | mp-1008985 |
| 27 | LiCoF2 | mp-1097040 | MgCr | mp-973060 | CoN | mp-1009078 |
| 28 | Ni(HO)2 | mp-27912 | Ni(HO)2 | mp-1180084 | FeHO2 | mp-755285 |
| 29 | LiCoO2 | mp-753473 | Co(HO)2 | mp-626708 | NiFeO2 | mp-1222302 |
| 30 | NiO2 | mp-35925 | Ni(HO)2 | mp-27912 | LiFeO2 | mp-19410 |
| 31 | LiCoN | mp-1246462 | VO | mp-19184 | Co [symbol] O2 | mp-1182397 |
| 32 | Li2CoN2 | mp-1247124 | Be4 [symbol] | mp-1227272 | LiFeOf | mp-775022 |
| 33 | Li3Co2NiO6 | mp-1222507 | FeO2 | mp-1062652 | LiNiO2 | mp-25411 |
| 34 | Li2FeNiO4 | mp-755008 | LiCoF2 | mp-1097040 | NiHO2 | mp-1067482 |
| 35 | NiO2 | mp-510753 | LiFeO2 | mp-19419 | Li4Co(O [symbol] )2 | mp-850355 |
| 36 | FeCuO2 | mp-510281 | Na2NiO2 | mp-752558 | NiHO2 | mp- [symbol] 7 |
| 37 | LiFeN | mp-1245817 | Li2CuO2 | mp-4711 | NiNiO2 | mp-25587 |
| 38 | CoO2 | mp-1077196 | Li2CoO2 | mp-755297 | LiNiO2 | mp-25316 |
| 39 | NaCuC2 | mp-579806 | MgCr | mp-1185898 | LiFeO3 | mp-1185320 |
| 40 | Li2NiO2 | mp-19183 | [symbol] 2CO | mp-1219429 | LiFeN | mp-1245817 |
| 41 | LiMoO2 | mp-19338 | MnBO3 | mp-1185996 | CoNF3 | mp-1213745 |
| 42 | Li(CoO2)2 | mp-552024 | VN | mp-1001826 | LiNiO3 | mp-1185281 |
| 43 | Li3Co(NiO3)2 | mp-122252 | NiHO2 | mp-999337 | Li4FeN2 | mp-28637 |
| 44 | Co(HO)2 | mp-24105 | CrO | mp-19091 | LiNiN | mp-29719 |
| 45 | CoO | mp-19128 | Ni(HO)2 | mp-625072 | Be3Fe | mp-963590 |
| 46 | LiCuOF | mp-1147631 | VN | mp-925 | NiO3 | mp-1209029 |
| 47 | CoC2 | mp-656750 | G [symbol] N2F3 | mp-1224894 | Be5Fe | mp-1025010 |
| 48 | Li2NiO2 | mp-19308 | Fe(HO)2 | mp-626680 | LisFeO2 | mp-765094 |
| 49 | LiMnO2 | mp-26373 | CrN | mp-1018157 | Be12Fe | mp-1104104 |
| 50 | LiCoN | mp-1147646 | VN | mp-1018027 | FeB2 | mp-509376 |

[symbol]

indicates data missing or illegible when filed

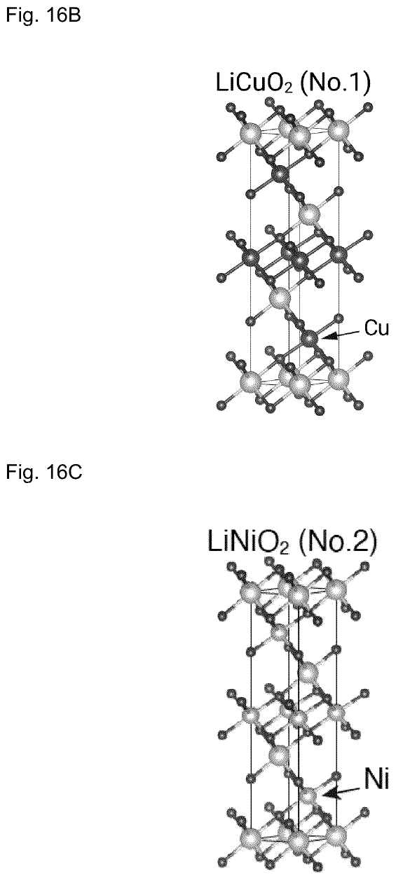

Table 2 shows materials extracted in the first to fiftieth proximities with respect to the query “LiCoO₂” according to the first experimental example and each comparative example. FIG. 16A illustrates the composition of the query “LiCoO₂”. FIG. 16B illustrates the composition of the material “LiCuO₂” extracted in the closest (first) proximity according to the first experimental example. FIG. 16C illustrates the composition of the material “LiNiO₂” extracted in the second proximity according to the first experimental example.

The query “LiCoO₂” is one of the most important cathode materials for a lithium-ion battery. In the first experimental example, the materials “LiCuO₂” and “LiNiO₂”, which each have the same layered structure as the query but contain different transition metal elements, were extracted in the first proximity and the second proximity of the query (cf. FIGS. 16A to 16C). In the first example, the materials extracted up to the seventh proximity had the same layered structure as the query, but contained different transition metal elements. These proximity materials included “LiNiO₂” and “LiFeO₂”, which is actually important lithium-ion battery materials. That is, the first example enabled the extraction of other important lithium-ion battery materials from “LiCoO₂”. In addition, in Example 1, most of the materials extracted up to the fiftieth proximity were lithium oxide. In contrast, in the methods of the respective comparative examples, inconsistent materials were extracted.

### (C) Summary

The analysis results of the two methods described above showed that the characteristic similarity of the material can be evaluated based on the positional relationship on the feature space where mapping is performed by the trained encoder even though information indicating the characteristic such as the structure of the material is not provided. That is, it was found that according to the machine learning, it is possible to generate a trained encoder that has acquired the ability to map data regarding the crystal structure onto a feature space where the characteristic similarity of a material can be found without providing information indicating the characteristic of the material. As a result, it was found that the trained encoder may provide new insights, such as characteristics of a new material and the search for a promising alternative material.

### (2) Second Experimental Example

A trained first encoder and a trained second encoder according to a second experimental example were generated under the same conditions as in the first experimental example except that the number of materials used for training was changed to 98,035 (80% of all data). By using the generated trained first encoder, the first data for each of 24,508 materials (20% of the total data) not used for training was converted into a first feature vector, and a map similar to that of the first experimental example was generated. The generated trained second encoder was also used to convert the second data for each of the 24,508 materials into a second feature vector. Then, by using the obtained second feature vector of each material as a query, an element (material) in the proximity of the query was extracted in the generated first feature vector map. As a result, it was evaluated whether or not the same material as the query using the second feature vector can be searched on the first feature vector map.

As a result of the evaluation, the probability of the same material being extracted in the first place was 56.628%. The probability of the same material being extracted up to the fifth place was 95.203%. The probability of the same material being extracted up to the tenth place was 99.078%. When elements are randomly extracted on the obtained map, the probability of the same material being extracted is 0.0041% (1/24,508). Hence it was found that the trained first encoder and the trained second encoder generated by the machine learning enable each of the first data and the second data for the same material to be mapped to the proximity range with high probability. That is, it was found that the first feature vector and the second feature vector of the same material have similar values are replaceable. This result showed the generation of a trained decoder corresponding to each encoder enables the generation of one of the first data and the second data from the other without greatly impairing the information.

### (3) Supplement

In each experimental example, three-dimensional atomic position data was adopted as the first data, and X-ray diffraction data was adopted as the second data. The three-dimensional atomic position data is a type of data indicating information regarding the local structure of the crystal of the material. The X-ray diffraction data is a type of data indicating information regarding the periodicity of the crystal structure of the material. Therefore, it was presumed that a result similar to the above could be obtained even if data indicating information regarding the local structure of the crystal of the material, other than the three-dimensional atomic position data, was adopted as the first data, and even if data indicating information regarding the periodicity of the crystal structure of the material, other than the X-ray diffraction data, was adopted as the second data. Examples of other data indicating the information regarding the local structure of the crystal of the material include Raman spectroscopy data, nuclear magnetic resonance spectroscopy data, infrared spectroscopy data, mass spectrometry data, and X-ray absorption spectroscopy data. Examples of other data indicating the information regarding the periodicity of the crystal structure of the material include neutron diffraction data, electron beam diffraction data, and total scattering data.

In addition, it is possible to evaluate the property of the material not necessarily based on both the local and overhead perspectives of the crystal structure. Therefore, it has been estimated that a result similar to the above may be obtained even if data indicating information regarding the local structure of the crystal of the material is not adopted as the first data or data indicating information regarding the periodicity of the crystal structure of the material is not adopted as the second data as long as the first data and the second data indicate properties of the material with indices different from each other.

### DESCRIPTION OF SYMBOLS

1 model generation device
11 controller
12 storage
13 communication interface
14 external interface
15 input device
16 output device
17 drive
81 generation program
91 storage medium
111 learning data acquisition unit
112 machine learning unit
113 storage processing unit
125 learning result data
2 data processing device
21 controller
22 storage
23 communication interface
24 external interface
25 input device
26 output device
27 drive
82 data processing program
92 storage medium
211 target data acquisition unit
212 conversion unit
213 restoration unit
214 estimation unit
215 output processing unit
31 first data
32 second data
35 correct information
41 first feature vector
42 second feature vector
51 first encoder
52 second encoder
55 first decoder
56 second decoder
58 estimator
61 first data
62 second data
71 first feature vector
72 second feature vector
63 first data
64 second data
73 first feature vector
65 second data
66 first data
75 second feature vector
67 first data
68 second data
77 first feature vector
78 second feature vector

1. A model generation method comprising:
a step of acquiring, by a computer, first data and second data regarding a crystal structure of a material, in which
the second data indicates a property of the material with an index different from an index of the first data,
the first data and the second data acquired include a positive sample and a negative sample,
the positive sample includes a combination of first data and second data for the same material, and
the negative sample includes at least one of first data and second data for a material different from the material of the positive sample; and
a step of performing, by the computer, machine learning for a first encoder and a second encoder by using the first data and the second data acquired, in which
the first encoder is configured to convert the first data into a first feature vector,
the second encoder is configured to convert the second data into a second feature vector,
a dimension of the first feature vector is the same as a dimension of the second feature vector, and
the machine learning for the first encoder and the second encoder is configured by training the first encoder and the second encoder so that values of a first feature vector and a second feature vector calculated from the first data and the second data of the positive sample are positioned close to each other, and a value of at least one of a first feature vector and a second feature vector calculated from at least one of the first data and the second data of the negative sample is positioned far from a value of at least one of the first feature vector and the second feature vector calculated from the positive sample.

2. The model generation method according to claim 1, further comprising the step of performing, by the computer, machine learning for a first decoder,
wherein the machine learning for the first decoder is configured by training the first decoder so that a result of the first decoder restoring the first data from a first feature vector, calculated from the first data by using the first encoder, matches the first data.

3. The model generation method according to claim 1, further comprising the step of performing, by the computer, machine learning for a second decoder,
wherein the machine learning for the second decoder is configured by training the second decoder so that a result of restoring the second data by the second decoder from a second feature vector, calculated from the second data by using the second encoder, matches the second data.

4. The model generation method according to claim 1, further comprising the step of performing, by the computer, machine learning for an estimator,
wherein
in the step of acquiring the first data and the second data, the computer further acquires correct information indicating a characteristic of the material, and
the machine learning for the estimator is configured by training the estimator, using the first encoder and the second encoder, so that a result of estimating the characteristic of the material from at least one of the first feature vector and the second feature vector, calculated from the first data and the second data acquired, matches the correct information.

5. The model generation method according to claim 1, wherein
the first data indicates information regarding a local structure of a crystal of the material, and
the second data indicates information regarding periodicity of a crystal structure of the material.

6. The model generation method according to claim 5, wherein the first data includes at least one of three-dimensional atomic position data, Raman spectroscopy data, nuclear magnetic resonance spectroscopy data, infrared spectroscopy data, mass spectrometry data, and X-ray absorption spectroscopy data.

7. The model generation method according to claim 5, wherein
the first data includes three-dimensional atomic position data, and
a state of an atom in the material is expressed by at least one of a probability density function, a probability distribution function, and a probability mass function in the three-dimensional atomic position data.

8. The model generation method according to claim 5, wherein the second data includes at least one of X-ray diffraction data, neutron diffraction data, electron beam diffraction data, and total scattering data.

9-13. (canceled)

14. An estimation method comprising the steps of:
acquiring, by a computer, at least one of first data and second data regarding a crystal structure of a target material;
converting, by the computer, at least one of the first data and second data acquired into at least one of a first feature vector and a second feature vector by using at least one of a trained first encoder and a trained second encoder; and
estimating, by the computer, a characteristic of the target material from a value of at least one of the obtained first feature vector and second feature vector by using a trained estimator,
wherein
the second data indicates a property of a material with an index different from an index of the first data,
a dimension of the first feature vector is the same as a dimension of the second feature vector,
the trained first encoder and the trained second encoder are generated by machine learning using first data and second data for learning,
the first data and the second data for learning include a positive sample and a negative sample,
the positive sample includes a combination of first data and second data for the same material,
the negative sample includes at least one of first data and second data for a material different from the material of the positive sample,
machine learning for the first encoder and the second encoder is performed by training the first encoder and the second encoder so that values of a first feature vector and a second feature vector calculated from the first data and the second data of the positive sample are positioned close to each other, and a value of at least one of a first feature vector and a second feature vector calculated from at least one of the first data and the second data of the negative sample is positioned far from a value of at least one of the first feature vector and the second feature vector calculated from the positive sample,
the trained estimator is generated by machine learning further using correct information indicating a characteristic of a material for learning, and
the machine learning for the estimator is configured by training the estimator so that a result of estimating the characteristic of the material for learning from at least one of the first feature vector and the second feature vector, calculated from at least one of the first data and the second data for learning by using at least one of the first encoder and the second encoder, matches the correct information.

15-17. (canceled)

18. An estimation device comprising:
a target data acquisition unit configured to acquire at least one of first data and second data regarding a crystal structure of a target material;
a conversion unit configured to convert at least one of the first data and second data acquired into at least one of a first feature vector and a second feature vector by using at least one of a trained first encoder and a trained second encoder; and
an estimation unit configured to estimate a characteristic of the target material from a value of at least one of the obtained first feature vector and second feature vector by using a trained estimator,
wherein
the second data indicates a property of a material with an index different from an index of the first data,
a dimension of the first feature vector is the same as a dimension of the second feature vector,
the trained first encoder and the trained second encoder are generated by machine learning using first data and second data for learning,
the first data and the second data for learning include a positive sample and a negative sample,
the positive sample includes a combination of first data and second data for the same material,
the negative sample includes at least one of first data and second data for a material different from the material of the positive sample,
machine learning for the first encoder and the second encoder is performed by training the first encoder and the second encoder so that values of a first feature vector and a second feature vector calculated from the first data and the second data of the positive sample are positioned close to each other, and a value of at least one of a first feature vector and a second feature vector calculated from at least one of the first data and the second data of the negative sample is positioned far from a value of at least one of the first feature vector and the second feature vector calculated from the positive sample,
the trained estimator is generated by machine learning further using correct information indicating a characteristic of a material for learning, and
the machine learning for the estimator is configured by training the estimator so that a result of estimating a characteristic of material for learning from at least one of the first feature vector and the second feature vector, calculated from at least one of the first data and the second data for learning by using at least one of the first encoder and the second encoder, matches the correct information.
